SEQUENCE LISTING (6 sequences)

SEQ ID NO: 1 - DNA, 17 bp, Artificial Sequence
  Synthetic
aggtggcccggcagaag

SEQ ID NO: 2 - DNA, 26 bp, Artificial Sequence
  Synthetic
gccttagttgcagtagttctccagct

SEQ ID NO: 3 - DNA, 21 bp, Artificial Sequence
  Synthetic
ccagaggcgcatgaagctaat

SEQ ID NO: 4 - DNA, 20 bp, Artificial Sequence
  Synthetic
cggcctctcgtcctgaccat

SEQ ID NO: 5 - DNA, 22 bp, Artificial Sequence
  Synthetic
aaccgaaaagccattgtagaaa

SEQ ID NO: 6 - DNA, 17 bp, Artificial Sequence
  Synthetic
cctggcgttgggattgg

(19) United States
(12) Patent Application Publication
(10) Pub. No.: US 2010/0305309 A1
(43) Pub. Date: Dec. 2, 2010

(54) NANODIAMOND PARTICLE COMPLEXES

(75) Inventors: Dean Ho, Chicago, IL (US); Mark Chen, Chicago, IL (US); Erik Pierstorff, Falls Church, VA (US); Erik Robinson, Chicago, IL (US); Robert Lam, Evanston, IL (US); Rafael Shimkunas, Palo Alto, CA (US); Xueqing Zhang, Evanston, IL (US)

Correspondence Address: Casimir Jones, S.C., 2275 DEMING WAY, SUITE 310, MIDDLETON, WI, 53562, US

(73) Assignee: Northwestern University, Evanston, IL (US)

(21) Appl. No.: 12/789,966

(22) Filed: May 28, 2010

Related U.S. Application Data
(60) Provisional application No. 61/181,993, filed on May 28, 2009.

(51) Int. Cl. C07K 17/14; C07H 21/00; C07C 237/26

(52) U.S. Cl. 530/402; 536/22.1; 552/203; 977/773

(57) ABSTRACT

The present invention provides various functionalized nanodiamond particles. In particular, the present invention provides soluble complexes of nanodiamond particles and therapeutic agents, for example insoluble therapeutics, anthracycline and/or tetracycline compounds, nucleic acids, proteins, etc.

### CROSS-REFERENCE TO RELATED APPLICATION

The present invention claims priority to U.S. Provisional Patent Application Ser. Nos. 61/181,993 filed May 28, 2009, which is hereby incorporated by reference in its entirety.

### STATEMENT REGARDING FEDERALLY SPONSORED RESEARCH OR DEVELOPMENT

This invention was made with government support under Grant Nos. CMMI-0846323, CMMI-0856492, and DMI-0327077 (Subcontract from the University of California-Berkeley, Subcontract Number SA5880-21593) from the National Science Foundation, and Grant No. U54 AI065359 from the National Institutes of Health. The government has certain rights in the invention.

### FIELD OF THE INVENTION

The present invention provides various functionalized nanodiamond particles. In some embodiments, the present invention provides complexes composed of nanodiamond particles and therapeutic agents. In certain embodiments, the present invention provides soluble complexes composed of nanodiamond particles and therapeutic agents that are water-insoluble or poorly water soluble. In some embodiments, the present invention provides complexes comprising nanodiamond particles and anthracycline and/or tetracycline compounds. In other embodiments, the present invention provides nanodiamond-nucleic complexes composed of polyethyleneimine surface functionalized nanodiamond particles and nucleic acid molecules. In further embodiments, the present invention provides alkaline-sensitive nanodiamond-protein complexes composed of nanodiamond particles and a protein adsorbed to the nanodiamond particles, where the protein is configured to desorb from the nanodiamond particles under sufficiently alkaline conditions.

### BACKGROUND

The application of nanoparticles as effective drug delivery vehicles, as well as in mechanical, electrical and MEMS applications has been demonstrated with carbon nanotubes, nanodiamonds, nanoparticle-embedded films, natural and synthetic polymers, lipid vesicles and a host of other nanoscale species [8, 9, 17-27]. Of these, detonated nanodiamonds are of interest primarily due to their small molecule loading capabilities [9, 28], functionalized surface [29] and biocompatibility [15, 30-32]. These attributes create a dynamic interface where the interactions between NDs and other particles or molecules can be defined by ND surface characteristics. An example of such an interaction is given by the supplied NDs possessing hydrophilic hydroxyl and carboxylic functional groups owing to characteristic surface charges and allowing for dispersion in water [8, 28, 29]. The future prospects of NDs in biomedical applications and their suggested biocompatibility manifests NDs as a favorable carbon-based biomaterial.

### SUMMARY OF THE INVENTION

The present invention provides various functionalized nanodiamond particles. In some embodiments, the present invention provides complexes composed of nanodiamond particles and therapeutic agents. In certain embodiments, the present invention provides complexes composed of nanodiamond particles and therapeutic agents that are water-soluble, water-insoluble, or poorly water soluble. In certain embodiments, the present invention provides soluble complexes composed of nanodiamond particles and therapeutic agents that are water-insoluble or poorly water soluble. In some embodiments, nanodiamond particles exhibit high binding capacity for one or more therapeutic agents. In other embodiments, the present invention provides nanodiamond-nucleic complexes composed of polyethyleneimine surface functionalized nanodiamond particles and nucleic acid molecules. In further embodiments, the present invention provides alkaline-sensitive nanodiamond-protein complexes composed of nanodiamond particles and a protein adsorbed to the nanodiamond particles, where the protein is configured to desorb from the nanodiamond particles under sufficiently alkaline conditions.

In some embodiments, the present invention provides compositions comprising a soluble complex, wherein the soluble complex comprises: a) a nanodiamond particle comprising one or more surface carboxyl groups; and b) a therapeutic agent, wherein the therapeutic agent is inherently water-insoluble or poorly water soluble (e.g., hydrophobic), wherein the therapeutic agent is adsorbed to the nanodiamond particle to form the soluble complex, and wherein the soluble complex is soluble in water (e.g., soluble in biological fluids, such as inside the human body) and suitable for in vivo administration to a human. In certain embodiments, the present invention provides compositions comprising a therapeutic agent adsorbed to a nanodiamond particle, wherein the nanodiamond particle comprises one or more surface carboxyl groups, wherein the therapeutic agent is water-insoluble or poorly water soluble when not adsorbed to the nanodiamond particle, and wherein the therapeutic agent is water soluble when adsorbed to the nanodiamond particle.

In some embodiments, the present invention provides compositions comprising a complex, wherein the complex comprises: a) a nanodiamond particle; and b) a therapeutic agent. In some embodiments, a therapeutic agent comprises a tetracycline class therapeutic. In some embodiments, a therapeutic agent comprises an anthracycline class therapeutic. In some embodiments, a therapeutic agent comprises one or more of daunorubicin, epirubicin, idarubicin, minocycline, tetracycline, oxytetracycline. In some embodiments, a therapeutic agent comprises one or more of daunorubicin, doxorubicin, epirubicin, idarubicin, valrubicin, mitoxantrone, tetracycline, chlortetracycline, oxytetracycline, demeclocycline, doxycycline, lymecycline, meclocycline, methacycline, minocycline, and/or rolitetracycline.

In other embodiments, the present invention provides methods of making a soluble complex comprising: mixing a nanodiamond particle with a therapeutic agent in the presence of an acid solution such that the therapeutic agent adsorbs to the nanodiamond particle thereby forming a soluble complex, wherein the therapeutic agent is inherently water-insoluble or poorly water soluble. In particular embodiments, the acid solution comprises acetic acid.

In some embodiments, the present invention provides compositions comprising a nanodiamond-nucleic acid complex, wherein the complex comprises: a) functionalized nanodiamond particles comprising one or more surface polyethyleneimine molecules; and b) nucleic acid molecules, wherein the nucleic acid molecules and the functionalized nanodiamond particles form a nanodiamond-nucleic acid complex.

In certain embodiments, the present invention provides methods of making a nanodiamond-nucleic acid complex comprising: a) mixing nanodiamond particles with polyethyleneimine molecules to generate functionalized nanodiamond particles; and b) mixing the functionalized nanodiamond particles with nucleic acid to generate a nanodiamond-nucleic acid complex.

In particular embodiments, the functionalized nanodiamond particles and the nucleic acid molecules form the nanodaimond-nucleic acid complex via attraction of positive charges on the functionalized nanodiamond particles and negative charges on the nucleic acid molecules. In other embodiments, the nucleic acid comprises DNA, RNA, a gene of interest, a microRNA, siRNA, or a plasmid. In particular embodiments, the nucleic acid molecules in the nanodiamond-nucleic acid complex are attached to the nanodiamond particles such that they are released upon cellular introduction. In certain embodiments, polyethyleneimine molecules are low molecular weight polyethyleneimine molecules.

In some embodiments, the present invention provides compositions comprising an alkaline-sensitive nanodiamond-protein complex, wherein the alkaline-sensitive nanodiamond complex comprises: a) a nanodiamond particle comprising one or more surface carboxyl or hydroxyl groups; and b) a protein (e.g., human insulin or other therapeutic protein), wherein the protein is adsorbed to the nanodiamond particle to form the alkaline-sensitive nanodiamond-protein complex, and wherein the protein is configured to desorb from the nanodiamond particle only under sufficiently alkaline conditions. In particular embodiments, the alkaline conditions are a pH of at least 8.0 . . . 8.5 . . . 9.0 . . . 9.5 . . . 10.0 . . . 10.5 . . . 11.0 . . . 12.0 . . . 13.0 . . . or 14.0.

In additional embodiments, the present invention provides methods of treating a subject comprising; a) providing: i) a subject comprising a treatment site that has an alkaline pH; and ii) a composition comprising an alkaline-sensitive nanodiamond complex, wherein the alkaline-sensitive nanodiamond complex comprises: A) a nanodiamond particle comprising one or more surface carboxyl or hydroxyl groups; and B) a protein, wherein the protein is adsorbed to the nanodiamond particle to form the alkaline-sensitive nanodiamond-protein complex; and b) administering (e.g., systemically, topically, orally, etc.) the composition to a subject under conditions such that: i) the alkaline-sensitive nanodiamond complex reaches the treatment site, and ii) the protein desorbs from the alkaline-sensitive nanodiamond complex in response to the alkaline pH at the treatment site. In particular embodiments, the alkaline conditions are a pH of at least 8.0 . . . 8.5 . . . 9.0 . . . 9.5 . . . 10.0 . . . 10.5 . . . 11.0 . . . 12.0 . . . 13.0 . . . or 14.0. In other embodiments, the treatment site is a wound and the administering is topical. In some embodiments, the protein comprises insulin (e.g., human insulin).

### DESCRIPTION OF THE DRAWINGS

FIG. 1. NDs enhance the ability to disperse Purvalanol A and 4-OHT in water. Vials were prepared against background and the reduction in turbidity mediated by the NDs was confirmed under the following conditions: A) 1 mg/ml ND in 5% DMSO in water; B) 1 mg/ml ND, 0.1 mg/ml Purvalanol A in 5% DMSO in water; C) 0.1 mg/ml Purvalanol A in 5% DMSO in water; D) 1 mg/mL ND in 25% DMSO in water; E) 1 mg/mL ND, 0.1 mg/mL 4-OHT in 25% DMSO in water; F) 0.1 mg/mL 4-OHT in 25% DMSO in water. G) TEM image of pristine NDs. H) 4-OHT residue can be observed on the ND surface to confirm ND-drug interactions. Scale bars represent 10 nm.

FIG. 2. UV-Vis spectrophotometric analysis of ND:4-OHT and Dex-ND complex pulldown. A) UV-Vis analysis of ND samples after centrifugation revealed a decrease in UV-Vis absorbance, confirming the ability to utilize the NDs as agents to interface with the 4-OHT and draw the drug into the pelleted ND sample. B) A comparative plot between the UV/Vis absorbance of 4-OHT and ND:4-OHT demonstrates ND and 4-OHT interfacing. The free 4-OHT in solution decreased as a result of physisorption to NDs, which were removed from the aqueous solution via centrifugation. Note there is no observed effect on separating 4-OHT from the aqueous supernatant phase when NDs are absent as illustrated by the overlapping dotted black and solid black lines representing 4-OHT before and after centrifugation, respectively. C) Dex-ND complex formation was also confirmed as shown by the sequestering of Dex following centrifugation.

FIG. 3. DLS analysis of particle size and zeta potential of ND-drug complexes. (FIG. 3A-3C): Average particle size of all drugs decreased upon physisorption to NDs. (FIG. 3D-3F): The zeta potential of all samples became more positive upon complexing with NDs.

FIG. 4. Therapeutic biofunctionality assays confirm maintained drug activity upon enhanced dispersion via ND complexing. A) Preservation of Purvalanol A activity was confirmed via a DNA fragmentation assay with the following lane designations: A) DNA Marker; B) Negative control (nothing added); C) 5% DMSO in water solution; D) 1 mg/ml ND in 5% DMSO in water solution; E) 1 mg/ml ND, 0.1 mg/ml Purvalanol A in 5% DMSO in water solution; F) 0.1 mg/ml Purvalanol A in 5% DMSO in water solution. Lane E confirmed the potent activity of ND-Purvalanol A complexes. B) MTT cell viability assays were performed to confirm the preserved therapeutic activity of 4-OHT following complex formation with the NDs. The following conditions were examined: (−): negative control; (+): positive control, 7.5 ug/mL 4-OHT; ND: 75 ug/mL ND; ND:4-OHT: 75 ug/mL ND, 7.5 ug/mL 4-OHT. All conditions were in culture media containing 1.31 mM acetic acid. Comparison of cell viability levels between the positive control and ND:4-OHT samples demonstrate preserved 4-OHT potency when complexed to NDs. One representative experiment of three is shown.

FIG. 5. Schematic illustration of (A) amino-functionalized nanodiamonds and (B) low molecular weight polyethyleneimine (PEI800) modified nanodiamonds.

FIG. 6. Size (A) and Zeta potential (B) of nanodiamonds and functionalized E nanodiamonds before pDNA binding. The particles were suspended in deionized water at a concentration of 60 ug/ml; Size (C) and Zeta potential (D) of nanodiamonds and functionalized nanodiamonds after pDNA binding with a fixed concentration of 3 ug pDNA/ml. The size measurements were performed using the Zetasizer Nano ZS (Malvern, Worcestershire, United Kingdom) at 25° C. at a 173° scattering angle. The mean hydrodynamic diameter was determined by cumulative analysis. The zeta potential determinations were based on electrophoretic mobility of the particles in the aqueous medium, which was performed using folded capillary cells in automatic mode. Data are represented as the mean±standard deviation (n=2). (E) TEM image of ND-PEI800/DNA. Scale bar is 20 nm.

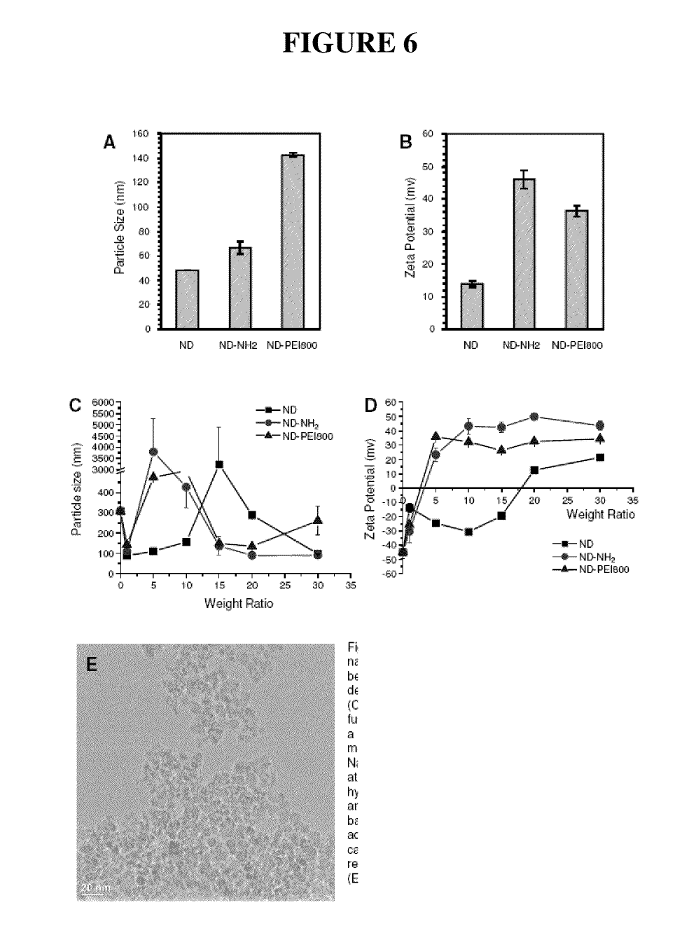

FIG. 7. PEI800 functionalized nanodiamonds mediated efficient gene transfection in HeLa cells. HeLa cells were seeded into 24-well plates at a density of 10⁵/well 24 h before transfection. Nanoparticles were added to the cells and incubated for 4 h at 37° C. Upon washing, cells were further incubated for 44 h. The concentrations of the particles were calculated on the basis of different weight ratios with a target pLuc dose of 3 μg/well. Cell harvesting and luciferase assays were performed 48 h after transfection. Data is represented as a mean±standard deviation (n=2). * represents particles with transfection efficiency lower than 10 RLU/mg of protein in the cell lysate.

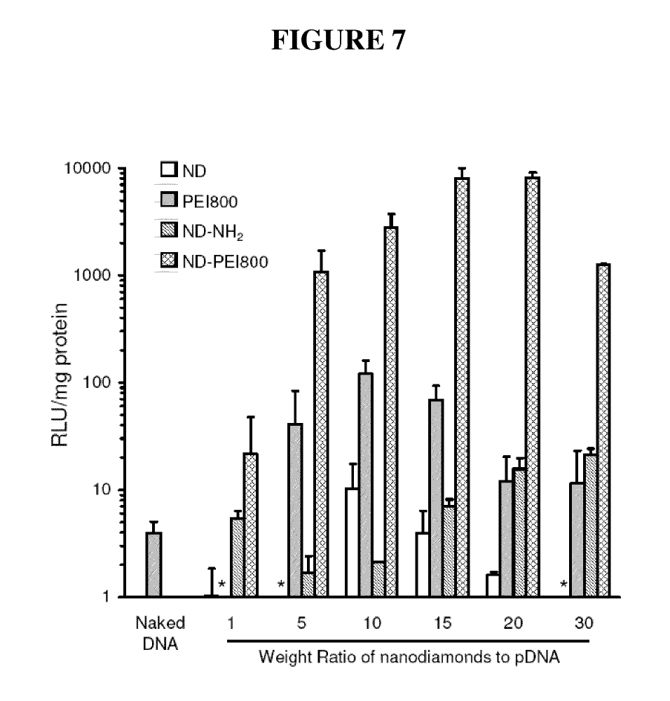

FIG. 8. Bright field and GFP confocal imaging of GFP expression in living HeLa cells mediated by ND-PEI800/pGFP at weight ratio of 5 (A) and 15 (B); unmodified nanodiamonds/pGFP at weight ratio of 5 (C) and 15 (D); PEI800/pGFP at weight ratio of 5 (E) and 15 (F); and naked pGFP (G). HeLa cells were seeded into 24-well plates at a density of 1.5×10⁵/well for 24 h before transfection. Nanoparticles were added to the cells and incubated for 4 h at 37° C. Upon washing, cells were further incubated for 44 h. The concentrations of the particles were calculated on the basis of a target pLuc dose of 6 μg/well. The living HeLa cells were washed by PBS and observed live under confocal microscropy (Leica Inverted Laser Scanning System, Argon Laser excitation 488 nm) 48 h after transfection. (Scale bar: 50 um)

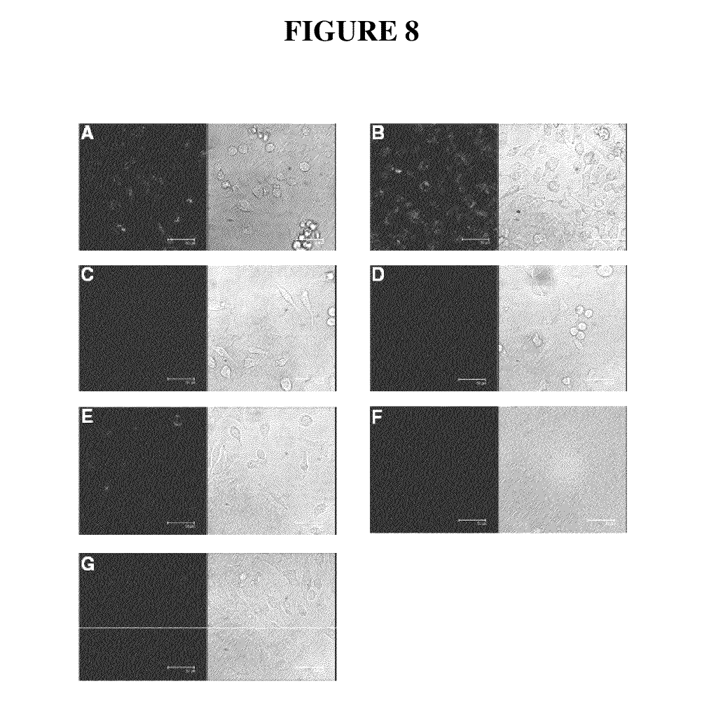

FIG. 9. A hypothetical schematic illustration showing insulin adsorption to NDs in water and desorption in the presence of NaOH. Insulin non-covalently binds to the ND surface in water by means of electrostatic and other interactions. The shift to an alkaline environment alters the insulin surface charge characteristics, thereby causing release from the ND surface.

FIG. 10. TEM images of (a) bare NDs, (b) NDs with adsorbed insulin in aqueous solution and (c) NDs with adsorbed insulin after treatment with 1 mM NaOH adjusted to pH 10.5. There is an apparent layer or coating on the surface of NDs (b), as compared to bare NDs (a), with a thickness of approximately 5-10 nm. Seeing as the addition of insulin was the only difference in sample preparation between (a) and (b), the visible layer may indicate insulin adsorption. The material is not present on the NaOH-treated NDs (c). The scale bar represents 20 nm in (a) and 50 nm in (b, c).

FIG. 11. Infrared spectra of (a) FITC insulin, (b) bare NDs and (c) ND-insulin complex. The arrows indicate the characteristic spectra of insulin present on the ND-insulin spectra, as compared to the bare-ND spectra. Image (c) suggests the formation of ND-insulin complexes as noted by the differential spectra. The data alludes to the non-covalent adsorption of insulin to NDs.

FIG. 12. Zeta potential changes associated with insulin and ND complexing at pH 7 and pH 10.5. NDs reveal a slightly positive zeta potential at both pH values, compared to the negative potential of insulin and the ND-insulin complex. The apparent difference in zeta potential between NDs and the ND-insulin complex implies an interaction between NDs and insulin.

FIG. 13. UV/vis quantification of the adsorption and desorption of insulin from NDs. (a) Adsorption of FITC insulin to NDs is noted by the differential absorbance values attained between the initial and centrifuged ND-insulin, measured at 485 nm. (b) Absorbance of bovine insulin implementing the BCA protein assay, measured at 562 nm. (c) Desorption of FITC insulin from NDs in 1 mM NaOH adjusted to various pH values. Samples were centrifuged under alkaline conditions, and the resultant solution measured. (d) Desorption of bovine insulin from NDs using the BCA protein assay. From the release absorbance spectra, greater amounts of insulin are desorbed in alkaline environments, suggesting NaOH affects the charge characteristics of insulin.

FIG. 14. Five-day insulin desorption test of ND-insulin samples treated with NaOH (pH 10.5) and water, showing insulin release in an alkaline pH environment. The cumulative weight percentage of released insulin was measured. The NaOH samples show increased desorption within the first 2 days and then a leveling-off of the amount released for a total desorption of 45.8±3.8%. Samples treated with water, however, released only a fraction of insulin totaling 2.2±1.2%. The majority of insulin released by NaOH occurred by day 1, indicating the alkaline solution had its maximal effect on fully-adsorbed NDs.

FIG. 15. MTT cell viability assay of RAW 264.7 macrophage cells under varying media conditions. Cells were serum-starved for 8 hours, followed by a 24-hour recovery period with the indicated media solutions: (1) DMEM, (2) 0.1 μM insulin, (3) 1 μM insulin, (4) approximately 0.1 μM insulin released from ND-insulin by NaOH (pH 10.5), (5) resultant solution from centrifuged ND-insulin in water, (6) ND-insulin treated with NaOH (1 μM total insulin), (7) NDs with bound insulin (ND-insulin, 1 μM total insulin) and (8) DMEM 10% FBS. Relative viability of ND-insulin treated with NaOH (6) was similar to that of high insulin concentration (3) demonstrating effective recovery with the released insulin. Insulin released by NaOH (4) showed higher relative viability than that of insulin released by water (5), signifying a greater desorption via alkaline solutions. ANOVA statistical analysis gave P<0.01, representing a significant difference among groups.

FIG. 16. (a) 3T3-L1 pre-adipocytes and (b) differentiated adipocytes, showing a clear difference in morphology between the two cell types. The pre-adipocyte fibroblast cells undergo differentiation upon supplementing media with insulin, dexamethasone and IBMX, becoming fully differentiated by day 10 post-induction. Lipid vesicle formation occurs during differentiation and can be seen in (b). 250× magnification.

FIG. 17. Real-time PCR gene expression for Ins1 and Csf3/G-csf under media conditions. 3T3-L1 adipocytes were serum-starved for 4 hours prior to a 2-hour recovery period in different media solutions: (1) DMEM, (2) 0.1 μM insulin, (3) 0.1 μM insulin released from ND-insulin by NaOH (pH 10.5), (4) resultant solution from centrifuged ND-insulin in water, (5) ND-insulin treated with NaOH (1 μM total insulin) and (6) NDs with bound insulin (ND-insulin, 1 μM total insulin). Both genes showed increased expression for insulin released by NaOH (3) and ND-insulin treated with NaOH (5), indicating effective insulin release by alkaline conditions while preserving activity. Comparatively, insulin released by water (4) and ND-insulin (6) demonstrated low relative expression for both genes alluding to the sequestration of insulin to the ND surface preventing protein function. Gene expression plot representative of RT-PCR samples. ANOVA: P<0.01.

FIG. 18. Spectroscopic analysis of Nanodiamond-Daunorubicin (ND-Daun) adsorption. Absorbance curves were measured before (BS) and after (AS) 15 minute spins (14000 rpm) to pellet any NDs or ND-Daun complexes present in each solution.

FIG. 19. Comparison of Nanodiamond-Daunorubicin (ND-Daun) adsorption. ND (1), Daun (2), ND-Daun (3), and ND-Daun+NaOH (4) solutions before (A) and after (B) 15 minute centrifugation at 14000 rpm.

FIG. 20. Desorption of DAUN from Nanodiamond conjugates in water and PBS respectively. Release profiles reveal drug elution is sustained over several hours. Absorbance measured at 485 nm.

FIG. 21. Spectroscopic analysis of Nanodiamond-Epirubicin (ND-Epi) adsorption. Absorbance curves were measured before (BS) and after (AS) 15 minute spins (14000 rpm) to pellet any NDs or ND-Epi complexes present in each solution.

FIG. 22. Comparison of Nanodiamond-Epirubicin (ND-Epi) adsorption. ND (1), Epi (2), ND-Epi (3), and ND-Epi+NaOH (4) solutions before (A) and after (B) 15 minute centrifugation at 14000 rpm.

FIG. 23. Desorption of EPI from Nanodiamond conjugates in water and PBS respectively. Release profiles reveal drug elution is sustained over several hours. Absorbance measured at 485 nm.

FIG. 24. Spectroscopic analysis of Nandiamond-Idarubicin (ND-IDA) adsorption. Absorbance curves were measured before (BS) and after (AS) two hour spins to pellet any NDs or ND-IDA complexes present in each solution.

FIG. 25. Comparison of ND-Ida adsorption. ND (1), Ida (2), ND-Ida (3), and ND-Ida+NaOH (4) solutions before (A) and after (B) 15 minute centrifugation at 14000 rpm.

FIG. 26. Desorption of IDA from Nanodiamond conjugates in water and PBS respectively. Release profiles reveal drug elution is sustained over several hours over. Absorbance measured at 485 nm.

FIG. 27. Spectroscopic analysis of ND-Daun-Dox-Epi-Ida adsorption. Absorbance curves were measured before (BS) and after (AS) 15 minute spins (14000 rpm) to pellet any NDs or ND-Daun+Dox+Epi+Ida complexes present in each solution.

FIG. 28. Comparison of ND-Daun-Dox-Epi-Ida adsorption. ND (1), Daun+Dox+Epi+Ida (2), ND-Daun+Dox+Epi+Ida (3), and ND-Daun+Dox+Epi+Ida+NaOH (4) solutions before (A) and after (B) 15 minute centrifugation at 14000 rpm.

FIG. 29. Spectroscopic analysis of Nanodiamond-Minocycline (ND-Mino) adsorption. Absorbance curves were measured before (BS) and after (AS) 15 minute spins (14000 rpm) to pellet any NDs or ND-Mino complexes present in each solution.

FIG. 30. Comparison of Nanodiamond-Minocycline (ND-Mino) adsorption. ND (1), Mino (2), ND-Mino (3), and ND-Mino+NaOH (4) solutions before (A) and after (B) 15 minute centrifugation at 14000 rpm.

FIG. 31. Desorption of Minocycline from Nanodiamond conjugates. Release profiles performed in water (above) and PBS (below) indicate sustained release over the course of the first few hours.

FIG. 32. Spectroscopic analysis of Nanodiamond-Tetracycline (ND-Tetra) adsorption. Absorbance curves were measured before (BS) and after (AS) 15 minute spins (14000 rpm) to pellet any NDs or ND-Tetra complexes present in each solution.

FIG. 33. Comparison of Nanodiamond-Tetracycline (ND-Tetra) adsorption. ND (1), Tetra (2), ND-Tetra (3), and ND-Tetra+NaOH (4) solutions before (A) and after (B) 15 minute centrifugation at 14000 rpm.

FIG. 34. Desorption of Tetracycline from Nanodiamond conjugates. Release profiles performed in water (above) and PBS (below) indicate sustained release over the course of the first few hours.

FIG. 35. Spectroscopic analysis of Nanodiamond Doxycycline (ND-Doxy) adsorption. Absorbance curves were measured before (BS) and after (AS) 15 minute spins (14000 rpm) to pellet any NDs or ND-Doxy complexes present in each solution.

FIG. 36. Comparison of Nanodiamond-Doxycycline (ND-Doxy) adsorption. ND (1), Doxy (2), ND-Doxy (3), and ND-Doxy+NaOH (4) solutions before (A) and after (B) 15 minute centrifugation at 14000 rpm.

FIG. 37. Desorption of Doxycycline from Nanodiamond conjugates. Release profiles performed in water (above) and PBS (below) indicate sustained release over the course of the first few hours.

FIG. 38. Spectroscopic analysis of Nanodiamond-Oxytetracycline (ND-Oxy) adsorption. Absorbance curves were measured before (BS) and after (AS) 15 minute spins (14000 rpm) to pellet any NDs or ND-Oxy complexes present in each solution.

FIG. 39. Comparison of Nanodiamond-Oxytetracycline (ND-Oxy) adsorption. ND (1), Oxy (2), ND-Oxy (3), and ND-Oxy+NaOH (4) solutions before (A) and after (B) 15 minute centrifugation at 14000 rpm.

### DETAILED DESCRIPTION

The present invention provides various functionalized nanodiamond particles. In certain embodiments, the present invention provides soluble complexes composed of nanodiamond particles and therapeutic agents that are water-insoluble or poorly water soluble. In some embodiments, the present invention provides complexes comprising nanodiamond particles and anthracycline and/or tetracycline compounds. In other embodiments, the present invention provides nanodiamond-nucleic complexes composed of polyethyleneimine surface functionalized nanodiamond particles and nucleic acid molecules. In further embodiments, the present invention provides alkaline-sensitive nanodiamond-protein complexes composed of nanodiamond particles and a protein adsorbed to the nanodiamond particles, where the protein is configured to desorb from the nanodiamond particles under sufficiently alkaline conditions.

### I. Nanodiamond-Drug Complexes

In some embodiments, the present invention provides complexes composed of nanodiamond particles and therapeutic agents. In certain embodiments, the present invention provides complexes of nanodiamond particles with therapeutic agents that are water-soluble, water-insoluble, or poorly water soluble. In certain embodiments, the present invention provides soluble complexes of nanodiamond particles with therapeutic agents that are water-insoluble or poorly water soluble. In some embodiments, the present invention provides complexes of nanodiamond particles with anthracycline- and/or tetracycline-class therapeutics (e.g. anthracycline, tetracycline, daunorubicin, epirubicin, idarubicin, minocycline, tetracycline, oxytetracycline, etc.). In some embodiments, nanodiamond particles exhibit high binding capacity for one or more therapeutic agents.

A broad array of water insoluble compounds have displayed therapeutically-relevant properties towards a spectrum of medical and physiological disorders including cancer and inflammation. However, the continued search for scalable, facile, and biocompatible routes toward mediating the dispersal of these compounds in water has limited their widespread application in medicine. Experiments performed during development of the present invention demonstrate a platform approach of water-dispersible, nanodiamond cluster-mediated interactions with several exemplary therapeutics to enhance their suspension in water with preserved functionality, thereby enabling novel treatment paradigms that were previously unrealized. These therapeutics include Purvalanol A, a highly promising compound for hepatocarcinoma (liver cancer) treatment; 4-Hydroxytamoxifen (4-OHT), an emerging drug for the treatment of breast cancer; and Dexamethasone, a clinically relevant anti-inflammatory that has addressed an entire spectrum of diseases that span complications from blood and brain cancers to rheumatic and renal disorders. Any water-insoluble or poorly water soluble therapeutic may be employed. Exemplary water insoluble agents include: for example: allopurinol, acetohexamide, benzthiazide, chlorpromazine, chlordiazepoxide, haloperidol, indomethacine, lorazepam, methoxsalen, methylprednisone, nifedipine, oxazepam, oxyphenbutazone, prednisone, prednisolone, pyrimethamine, phenindione, sulfisoxazole, sulfadiazine, temazepam, sulfamerazine, and/or trioxsalen. Water-insoluble, poorly water soluble, or lipid soluble therapeutics which find use in embodiments of the present invention include central nervous system drugs, peripheral nervous system drugs, sensory organ drugs, cardiovascular system drugs, respiratory system drugs, hormones, urogenital system drugs, drugs for anal diseases, vitamins, drugs for liver diseases, antigout drugs, enzymes, antidiabetics, immunosuppressants, cytoactivators, antitumoral drugs, radioactive drugs, antiallergic drugs, antibiotics, chemotherapeutic agents, biological drugs, and extracorporeal diagnostic agents. More particularly, water-insoluble, poorly water soluble, and/or lipid soluble therapeutics that find use in ND-complexes of the present invention include steroidal drugs (e.g. dexamethasone, prednisolone, betamethasone, beclomethasone propionate, triamcinolone, hydrocortisone, fludrocortisone and prasterone, salts thereof, and their lipid-soluble derivatives), β-adrenergic agonists (e.g. procaterol, orciprenaline, isoproterenol hydrochloride, pirbuterol, terbutaline, hexoprenaline, fenoterol hydrobromide, hexoprenaline sulfate, terbutaline sulfate, salbutamol sulfate, oxyprenaline sulfate, formoterol fumarate, isoprenaline hydrochloride, pirbuterol hydrochloride, procaterol hydrochloride, mabuterol hydrochloride, and tulobuterol, salts thereof, and their lipid-soluble derivatives), xanthine derivatives (e.g. diprophylline, proxyphylline, aminophylline and theophylline, salts thereof, and their lipid-soluble derivatives), antibiotics (e.g. pentamidine isethionate, cefmenoxime, kanamycin, fradiomycin, erythromycin, josamycin, tetracycline, minocycline, chloramphenicol, streptomycin, midecamycin, amphotericin B, itraconazole and nystatin, salts thereof, and their lipid-soluble derivatives), and therapeutics of other classes (e.g. ipratropium bromide, methylephedrine hydrochloride, trimethoquinol hydrochloride, clenbuterol hydrochloride, oxitropium bromide, fultropium bromide, methoxyphenamine hydrochloride, clorprenaline hydrochloride sodium cromoglycate, etc.). In some embodiments, a complex is based upon NDs and a combination of two or more of the above listed agents or other agents understood by those in the art (e.g. 2 therapeutic agents, 3 therapeutic agents, 4 therapeutic agents, 5 therapeutic agents . . . 10 therapeutic agents . . . 20 therapeutic agents, etc.). Given the scalability of nanodiamond processing and functionalization, this approach serves as a facile, broadly impacting and significant route to translate water-insoluble compounds towards treatment-relevant scenarios.

Many biomedically-relevant compounds are difficult to solubilize in water, thus limiting their therapeutic potential [1-5]. These compounds have displayed remarkable therapeutic properties in vitro towards diseases such as liver and breast cancer [1-2]. However, since these therapeutics are soluble primarily in solvents generally regarded as unsuitable for injection, the realization of new routes to patient treatment enabled by these drugs has been hindered. As there remains a widespread need to package these compounds for facile delivery, a spectrum of polymeric and carbon-based nanomaterials have been explored [6-15]. For example, block copolymer-stabilized nanoemulsions have recently been explored as vehicles for polar and nonpolar agents [6]. Furthermore, lipid-polymer hybrid nanoparticles comprised of lipid-PEG shells and a poly(lactic-co-glycolic acid) (PLGA) hydrophobic core have been developed for the release of drugs that are poorly water soluble [7]. With regards to carbon-based strategies for the dispersal of poorly water-soluble drugs, PEGylated nano-graphene oxides have recently been explored for the delivery of an aromatic camptothecin (CPT) analog [15].

Nanodiamonds (NDs) represent an important, emerging class of materials that possess several medically-significant properties [16-36]. To produce highly uniform particle diameters of 4-6 nm, NDs can be inexpensively processed via ultrasonication, centrifugation, and milling methodologies [22,26]. Furthermore, acid treatment to remove impurities can simultaneously result in carboxyl group surface functionalization which can be harnessed towards subsequent drug interfacing. In addition, surface-bound carboxyl groups enable stable ND suspension in water. Therefore, these streamlined processes provide a rapid, inexpensive, and highly efficient approach towards making NDs scalable materials for medicine. Previous studies of NDs have demonstrated their carrier capabilities with Doxorubicin, cellular internalization without the need to coat the NDs with biocompatible or lipophilic agents, and preservation of drug efficacy upon murine macrophage and human colon cancer cell lines. Furthermore, comprehensive biocompatibility assays using quantitative real-time polymerase chain reaction (RT-PCR) interrogation of inflammatory cytokines have revealed their biocompatible properties.²⁶ During development of embodiments of the present invention is has been shown that ND clusters are additionally capable of complexing with poorly water-soluble drugs to enhance their dispersive properties in water. To demonstrate the platform capabilities of the NDs, three drugs with important implications (Purvalanol A, 4-hydroxytamoxifen), or demonstrated relevance (Dexamethasone) served as model systems.

Nanodiamonds provide a platform for the facile solubilization of a broad range of small molecule, protein, antibody, and RNA/DNA therapies. The present invention is not limited by the therapeutic agent that is employed. Work conducted during development of embodiments of the present invention has shown that nanodiamond powder platforms can be applied towards the rapid water solubilization of a broad range of therapeutic compounds that are currently translationally challenged because of their insolubility in water alone (e.g. currently soluble in DMSO, Ethanol, all solvents which preclude human use). By adding a small amount of acid (e.g., 1% or less) during the functionalization/drug-linking process, which we have demonstrated the linking of compounds such as 4-hydroxytamoxifen (4-0HT, a Breast Cancer therapeutic soluble in Ethanol), Purvalanol A (Liver cancer therapeutic soluble in DMSO), and Dexamethasone (Anti-inflammatory soluble in ethanol/methanol). The acid functionalization process is not toxic to cells as shown by proliferation assays, and there is a very minute and brief change in pH that is rapidly restored to normal levels within a few hours. This is a highly scalable process given the very economical characteristics of nanodiamond production, purification, and functionalization. Furthermore, in certain embodiments, this is a one step process and can be completed in minutes, making this perhaps among the most scalable processes for the solubilization of water insoluble drugs. Given the vast array of already known and undiscovered compounds with transforrnative treatment potential, but prohibitive water insolubility, the present invention meets the goals of optimized drug solubilization by being biocompatible, economical/scalable, and very rapid in terms of processing speed.

Many potentially useful pharmaceuticals cannot be used for clinical application due to toxicity. In some embodiments, the present invention provides complexes composed of nanodiamond particles and toxic or potentially toxic therapeutic agents. In some embodiments, complexing the therapeutic agent to the nanodiamond particles reduces drug toxicity and renders the drug safe for clinical application.

In some embodiments, the present invention provides complexes of nanodiamond particles and vaccines. In some embodiments, the present invention provides delivery and sustained release of one or more vaccines into a subject. In some embodiments, release of vaccine from complexes of the present invention reduces side effects from vaccine delivery, and enhances efficiency of vaccine delivery. In some embodiments, vaccines which find use with the present invention include, but are not limited to: influenza vaccine, cholera vaccine, bubonic plague vaccine, polio vaccine, hepatitis A vaccine, rabies vaccine, yellow fever, measles/mumps/rubella, typhoid vaccine, tetanus vaccine, diphtheria vaccine, Mycobacterium tuberculosis vaccine, etc.

In some embodiments, the present invention provides complexes of nanodiamond particles and one or more antimicrobial agents. In some embodiments, the present invention provides delivery and sustained release of one or more antimicrobial agents into a subject. In some embodiments, release of antimicrobial agent from complexes of the present invention reduces side effects and enhances efficiency of antimcrobial delivery. In some embodiments, antimicrobial agents which find use with the present invention include, but are not limited to: antibiotics, antivirals, antifungals, and antiparasitics.

In some embodiments, the present invention provides complexes of nanodiamond particles and anthracycline- and/or tetracycline-class therapeutics (e.g. anthracycline, tetracycline, daunorubicin, epirubicin, idarubicin, minocycline, tetracycline, oxytetracycline, etc.). In some embodiments, anthracycline- and/or tetracycline-class therapeutics, or derivatives thereof, are water-insoluble or have poor solubility in water. In some embodiments, anthracycline- and/or tetracycline-class therapeutics, or depravities thereof, are water soluble. In some embodiments ND-anthracycline complexes and/or ND-tetracycline complexes exhibit remarkable binding capacity between the ND surface and therapeutic compounds. Experiments conducted during development of embodiments of the present invention demonstrate exceptional binding between the ND surface and therapeutic compounds in ND complexes with therapeutics including daunorubicin, epirubicin, idarubicin, minocycline, tetracycline, oxytetracycline. In some embodiments, complexes between NDs and one or more any suitable anthracycline- and/or tetracycline class therapeutic exhibit high binding capacity. In some embodiments, complexes are based upon NDs and one, or any combination, of anthracyclines (e.g. daunorubicin, doxorubicin, epirubicin, idarubicin, valrubicin, mitoxantrone, etc.) and tetracyclines (e.g. tetracycline, chlortetracycline, oxytetracycline, demeclocycline, doxycycline, lymecycline, meclocycline, methacycline, minocycline, rolitetracycline). Experiments conducted during development of embodiments of the present invention have demonstrated that ND/anthracycline-class complexes and/or ND/tetracycline-class complexes bind in a very tight fashion while remaining dispersed in water. Although the present invention is not limited to any particular mechanism of action and an understanding of the mechanism of action is not necessary to practice the present invention, it is contemplated that opposite charges between the surface of acid washed NDs and the therapeutic compounds result in high potency binding following NaOH or KOH treatment. In some embodiments, drug release from ND/anthracycline-class complexes and/or ND/tetracycline-class complexes occurs in a sustained fashion. Experiments conducted during development of embodiments of the present invention have demonstrated that for drug-resistant disease models (e.g. cancer), the very tight ND-drug binding allows the drug to be ferried into the cell, and resistance can be counter-acted as the NDs maintain intracellular drug presence. As such, drug ejection/efflux is prevented. In some embodiments, ND/anthracycline-class complexes and/or ND/tetracycline-class complexes provide effective treatment of multi-drug resistant diseases such (e.g. cancer, tuberculosis, bacterial infections, etc.). In some embodiments, ND/anthracycline-class complexes and/or ND/tetracycline-class complexes provide effective treatment of multi-drug resistant diseases such (e.g. cancer, tuberculosis, bacterial infections, etc.) because drug ejection/efflux from cells is prevented.

The present invention is generally applicable to an extremely broad spectrum of treatment strategies, from cancer, to inflammation, to regenerative medicine, etc. In some embodiments, the compositions and methods of the present invention provide treatment, symptom reduction and/or prevention of one or more diseases, indications, conditions, and disorders including, but not limited to: acute myeloid leukemia, drug-resistant leukemias, breast cancer, lymphomas, uterine cancers, lung cancer, ovarian cancer, malaria, veterinary applications, vancomycin-resistant enterococcus (VRE), Parkinsons (e.g. as a neuroprotective agent), fibromyalgia, infected animal bite wounds (e.g. pasteurella multocida, pasteurella pneumotropica, etc.), rheumatoid arthritis, reactive arthritis, chronic inflammatory lung diseases (e.g. panbronchiolitis, asthma, cystic fibrosis, bronchitis, etc.), sarcoidosis, prevention of aortic aneurysm in patients with Marfan Syndrome, multiple sclerosis, meibomian gland dysfunction, acne, amoebic dysentery, anthrax, cholera, gonorrhea (e.g. when penicillin cannot be given), Gougerot-Carteaud Syndrome, lyme disease, bubonic plague, periodontal disease, respiratory infections (e.g. pneumonia), HIV (e.g. as an adjuvant to HAART), Rocky Mountain spotted fever, syphilis (e.g. when penicillin cannot be given), urinary tract infections, rectal infections, infections of the cervix, upper respiratory tract infections (e.g. caused by Streptococcus pyogenes, Streptococcus pneumoniae and Hemophilus influenza), lower respiratory tract infections (e.g. caused by Streptococcus pyogenes, Streptococcus pneumoniae, Mycoplasma pneumonia, skin and soft tissue infections (e.g. caused by Streptococcus pyogenes, Staphylococcus aureaus), infections caused by rickettsia (e.g. Rocky Mountain spotted fever, typhus group infections, Q fever, rickettsialpox), Psittacosis of ornithosis (e.g. caused by Chlamydia psittaci), infections caused by Chlamydia trachomatis (e.g. uncomplicated urethral, endocervical, or rectal infections; inclusion conjunctivitis;trachoma; lymphogranuloma venereum, etc.), granuloma inquinale (e.g. caused by Calymmatobacterium granulomatis), relapsing fever (e.g. caused by Borrelia sp.), bartonellosis (e.g. caused by Bartonella bacilli-formis), chancroid (e.g. caused by Hemophilus ducreyi), tularemia (e.g. caused by Francisella tularensis), plaque (e.g. caused by Yersinia pestis), cholera (e.g. caused by Vibrio cholera), Campylobacter fetus infections, intestinal amebiasis (e.g. caused by Entamoeba histolytica), urinary tract infections (e.g. caused by susceptible strains of Escherichia coli, Klebsiella, etc.), infections caused by susceptible gram-negative organisms (e.g. E. coli, Enterobacter aerogenes, Shigella sp., Acinetobacter sp., Klebsiella sp., and Bacteroides sp.), severe acne, etc. In some embodiments, compositions and methods of the present invention are also relevant towards nonbiological processes that require the water solubilization of insoluble agents, especially when they can be rapidly coupled to an inert substance such as nanodiamonds that are very stable, and can be easily removed, if necessary, via simple centrifugation processes. For biological applications, it has been shown that nanodiamonds can be removed in vivo via the urinary system, confirming their bio-amenability.

### II. Nanodiamond-Nucleic Acid Complexes

The present invention provides nanodiamond-nucleic acid complexes that are capable of nucleic acid release with preserved function. In certain embodiments, such complexes serve as non-viral gene delivery vectors. Such ND-nucleic acid complexes may be employed, for example, in a broad array of medical disorders including cancer, inflammation, autoimmune diseases, wound healing, pain, neurological disorders, and other types of disorders. By functionalizing the ND surface with low molecular weight polyethyleneimine (e.g., PEI800), it was shown that DNA plasmids were capable of being released upon cellular introduction whereas without the functionalization step, the DNA could be bound (via physisorption) to the NDs, but not released. ND-nucleic acid complexes may be used, for example, in the treatment for cancer, inflammation, pain, scarring/wound healing, infection, and diabetes insulin delivery, and other disorders capable of treatment with gene therapy type approaches.

### III. Alkaline-Sensitive Nanodiamond-Protein Complexes

The present invention provides nanodiamond-protein complexes that allow, for example, desorption of the protein in alkaline environments. Work conducted during the development of embodiments of the present invention exemplified this invention with the development of a Nanodiamond(ND)-Insulin complex that is capable of pH-dependent protein release (e.g., for applications in diabetes treatment as well as wound healing). This is important as it has been shown that following skin burns, insulin is immediately administered to prevent infection, a major complication. Furthermore, it has been shown that skin pH levels following burns can reach basic levels (e.g. 10-11). Work conducted during the development of embodiments of the present invention has shown that such complexes can selectively release insulin at that pH level while unreleased insulin function is sequestered until it is delivered. In certain embodiments, such as those where the protein is insulin, the ND-protein complexes are use for the treatment of wound healing, infection, and diabetes insulin delivery, among others.

There remains a significant need for enhanced methods of drug delivery to maximize therapeutic effects while decreasing associated complications. Systemic treatments pose various problems concerning the pervasiveness of drug exposure to the body and can lead to harmful side effects outweighing treatment benefits. Effectively targeting and controlling drug delivery as to limit drug-tissue interaction is a desired outcome. In this regard, site-specific drug delivery is highly advantageous for a host of ailments ranging from cancer to cardiovascular treatments. Recent advances in nanomedicine (e.g., imaging and diagnosis [1-3], drug delivery [4-10] and gene therapy [11-13]) have demonstrated the benefits of nanoparticle therapeutics, including reduction of drug concentration, targeted delivery, diminished complications and biocompatibility [3, 14-16].

Numerous studies have shown the efficacy of transiently linking or conjugating drugs and therapeutic molecules to NDs, including chemotherapy agents, organic molecules and proteins [29, 33, 34]. There has been recent work concerning the drug release profiles of NDs [8, 9], yet there is little scientific inquiry relating to the release of protein-based drugs. Examples of protein-based drugs include cytokines, monoclonal antibodies, hormones and clotting factors, all of which hold great promise or have been substantiated for targeted drug delivery.

Enhanced specificity in drug delivery aims to improve upon systemic elution methods by locally concentrating therapeutic agents and reducing negative side effects. As described in Example 2 below, bovine insulin was non-covalently bound to detonated nanodiamonds via physical adsorption in an aqueous solution and demonstrated pH-dependent desorption in alkaline environments of sodium hydroxide. Insulin adsorption to NDs was confirmed by FT-IR spectroscopy and zeta potential measurements, while both adsorption and desorption were visualized with TEM imaging, quantified using protein detection assays and protein function demonstrated by MTT and RT-PCR. NDs combined with insulin at a 4:1 ratio showed 79.8±4.3% adsorption and 31.3±1.6% desorption in pH-neutral and alkaline solutions, respectively. Additionally, a 5-day desorption assay in NaOH (pH 10.5) and neutral solution resulted in 45.8±3.8% and 2.2±1.2% desorption, respectively. MTT viability assays and quantitative RT-PCR (expression of Ins1 and Csf3/G-csf genes) reveal bound insulin remains inactive until alkaline-mediated desorption. Thus, the present invention provides for applications in sustained drug release, wound therapy and imaging employing a therapeutic protein-ND complex with demonstrated tunable release and preserved activity.

### EXAMPLES

The following Examples are presented in order to provide certain exemplary embodiments of the present invention and are not intended to limit the scope thereof.

### Example 1

### Soluble Nanodiamond-Drug Complexes

This example describes the preparation and testing of soluble nanodiamond-drug complexes.

### ND-Drug Complex Preparation

Samples of NDs (20 mg/ml), ND:Purvalanol A (10:1 ratio-20 mg/ml ND, 2 mg/ml Purvalanol A), and Purvalanol A alone (2 mg/ml) suspended in DMSO were prepared. The DMSO mixtures were diluted 20 fold in water to create a 5% DMSO solution with the various mixtures of ND and drug.

To prepare the ND:4-OHT complexes, 1 mg 4-OHT was solubilized in 174 mM acetic acid in de-ionized water. NDs (10 mg/ml) were sonicated for 4 hours, added to the 4-OHT sample, and thoroughly vortexed to yield a ND:4-OHT conjugate solution (5 mg/mL ND, 0.5 mg/mL 4-OHT). Solvent only (174 mM acetic acid), ND only (5 mg/mL), and 4-OHT only (0.5 mg/mL) solutions were prepared as controls.

### UV-Vis Spectrophotometric Characterization of Drug Adsorption/Desorption

Prior to scanning, all samples were diluted to concentrations of 50 μg/mL and 500 μg/mL for 4-OHT and NDs, respectively. All samples underwent centrifugation at 14,000 rpm for 2 hours at 25° C., where the supernatant was then subsequently collected for spectroscopic scans from 200 nm to 600 nm. Drug loading concentrations were determined via ND-complex pull-down experiments which comprised of an initial absorbance reading, then a 2 hour centrifuge of all samples at 25° C. and 14000 RPM followed by a final absorbance reading. The concentration of loaded drug was then calculated by measuring the difference between the initial and final readings.

### Transmission Electron Microscopy

TEM was performed by sonicating the ND:4-OHT solution and then pipetting a droplet onto a carbon TEM grid (Ted Pella). Following 2 hours of drying, a JEOL 2100F Field Emission Gun TEM was used for high voltage 200 kV imaging. A pristine ND sample was also imaged via the same protocol.

### Particle Size and Zeta Potential Measurement

The particle size and zeta potential of the complexes were measured using a Zetasizer Nano (Malvern Instruments). ND:4-OHT and Dex-ND complexes were prepared in 25% aqueous DMSO as described previously. ND:Purvalanol A complexes were prepared in a similar manner in 5% aqueous DMSO as described previously. The final concentration of ND and therapeutic in all complexes was 1 mg/mL and 0.1 mg/mL, respectively. All size measurements were performed at 25° C. at a 90° scattering angle. Mean hydrodynamic diameters were obtained via cumulative analysis of 11 measurements. The zeta potential measurements were performed using capillary wells at 25° C., and the mean potential obtained via cumulative analysis of 15 measurements.

### DNA Fragmentation Assays

A 1:10 dilution of 5% DMSO in water, NDs in 5% DMSO in water (1 mg/ml), ND:Purvalanol A in 5% DMSO in water (10:1 ratio-1 mg/ml ND, 0.1 mg/ml Purvalanol A), and Purvalanol A in 5% DMSO in water (0.1 mg/ml) were added to HepG2 tissue culture cells and grown for 24 hours. The cultured cells were lysed in 500 μL lysis buffer (10 mM Tris-HCl, pH 8.0, 10 mM EDTA, 1% Triton X-100). 30-minute incubations at 37° C. followed separate RNase A and proteinase K treatment. Following phenol chloroform extraction, nuclear DNA was isolated in isopropyl alcohol and stored at −80° C. overnight. The samples were then resuspended in DEPC water following a 70% ethanol wash and electrophoresed using a 0.8% agarose gel, and finally stained with ethidium bromide.

### MTT Cell Viability Assay

MCF-7 cells were plated to 50% confluence in 96-well plates in pH 7.1 MEM/EBSS culture media containing 75 ug/mL NDs, or ND:4-OHT complexes (75 ug/mL ND, 7.5 ug/mL 4-OHT). 7.5 ug/mL 4-OHT was used as a positive control. All samples accounted for the 1.31 mM acetic acid associated with the 4-OHT ND complex solution. Cultures were maintained at 37° C., 5% CO₂ for 44 hours prior to performing the MTT-based cell viability assay according to the manufacturer's protocol (Sigma-Aldrich). Absorbances were determined at 570 nm using a Safire multiwell plate reader (Tecan) and Magellan software (Tecan). All samples were run in triplicate.

### Results

NDs were synthesized, purified, and processed as previously described [22,26]. Fourier transform infrared spectroscopy (FTIR) measurements confirmed the presence of carboxyl groups on the surface which were deposited as a result of acid treatment during the purification process to remove contaminants [26]. The utility of the carboxyl groups was initially hypothesized to contribute to the ability to interface the NDs with drug molecules through physisorption or electrostatic interactions such that the drug could eventually be released upon external stimuli. In this Example, this hypothesis was confirmed via a multitude of drug-ND imaging and characterization experiments, and UV-Vis analysis of drug-ND interfacing, in addition to functionality assays.

Due to its enormous potential as a chemotherapeutic for liver cancer, Purvalanol A was an ideal drug to complex with NDs. Soluble in DMSO, Purvalanol A is a cyclin dependent kinase inhibitor capable of interrupting cell cycle progression. It has been shown to promote death in cell lines that overexpress myc, an oncogene that is often constitutively expressed in cancers. Due to the role of myc in cell proliferation, its overexpression or mutation often leads to cancer. 4-hydroxytamoxifen (4-OHT), a water-insoluble breast cancer therapeutic, was selected as another model drug system due to its demonstrated efficacy against estrogen-relevant cancers. Lastly, Dexamethasone (Dex) was selected as an additional drug model due to its broad clinical relevance as a steroidal anti-inflammatory, among other physiological conditions toward which it is applicable. All ND-drug complexes were demonstrated to be rapidly dispersable in water, indicating the potential applicability of ND platforms as scalable, water-insoluble therapeutic compound delivery agents.

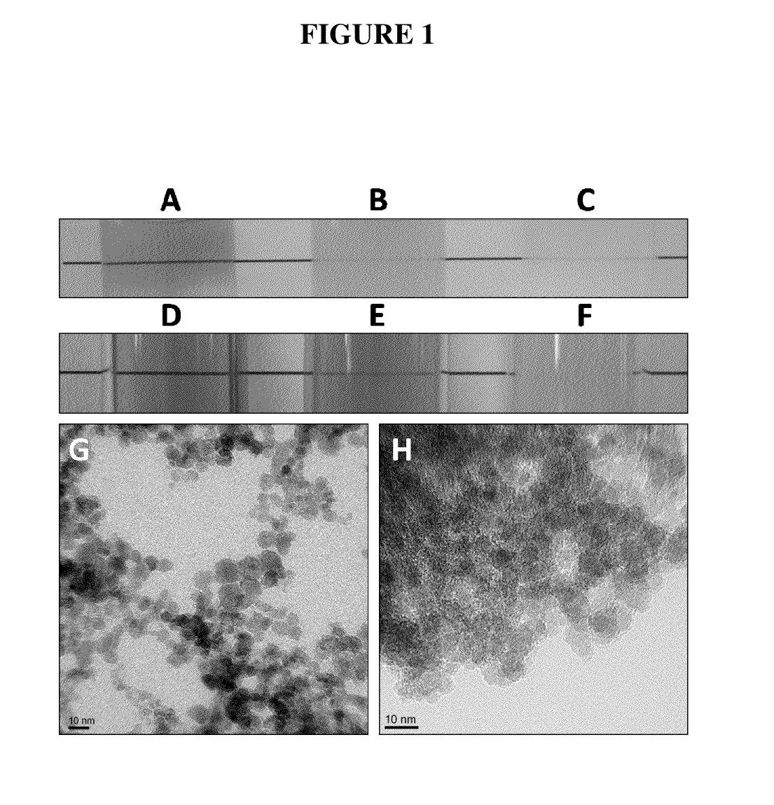

In order to examine the solubility changes with the introduction of NDs, samples of NDs (20 mg/ml), ND:Purvalanol A (10:1 ratio-20 mg/ml ND, 2 mg/ml Purvalanol A), and Purvalanol A alone (2 mg/ml) suspended in DMSO were compared. The DMSO mixtures were diluted 20 fold in water to create a 5% DMSO solution with the various mixtures of ND and drug (FIG. 1A-1C). Following dilution in water, Purvalanol A precipitated out of solution, producing a turbid liquid (FIG. 1C). The presence of NDs significantly reduced the turbidity of Purvalanol A aqueous solutions, presumably through efficient drug adsorption to the ND surface, which implies a reduction in free Purvalanol A in solution. This surface interface between the NDs and therapeutics has been confirmed for numerous types of drugs in this study (e.g. Doxorubicin, 4-hydroxytamoxifen, Dexamethasone, etc.). While not necessary to understand or practice the present invention, and while not limiting the present invention, it is hypothesized that physisorption is the main interaction between Purvalanol A and the NDs. It has been previously demonstrated the potential for small molecule release by modulating this interaction with the addition and removal of salts.²⁶ Due to the reversible nature of the Purvalanol A-ND interface, the complexes served as a favorable platform for both initially dispersing the drug in water and facilitating its subsequent release.

4-hydroxytamoxifen (4-OHT) was selected as the second therapeutic for ND-drug complexing given its importance as a triphenylethylene (TPE) treatment strategy for estrogen receptor (ER)-positive breast cancer. 4-OHT is soluble in ethanol and is often prescribed for its localized activity upon the breast even through systemic administration and therapy, which for other drugs can normally result in non-specific effects. 4-OHT administration has been shown to reduce the risk of local recurrence, by preventing introduction of new primary tumors to the breast [37-40].

ND-mediated enhancement of 4-OHT solubility in water was qualitatively examined and confirmed by observing degrees of visibility through vials which contained ND, 4-OHT, and ND:4-OHT samples in 25% DMSO similar to the interfacial test done with Purvalanol A (FIG. 1D-1F). In addition, to visually confirm ND:4-OHT interfacing, transmission electron microscope (TEM) images of NDs with and without bound 4-OHT were compared (FIG. 1G-1H). It was clearly observed that an amorphous 4-OHT residue was present upon drug addition to the ND solution (FIG. 1H).

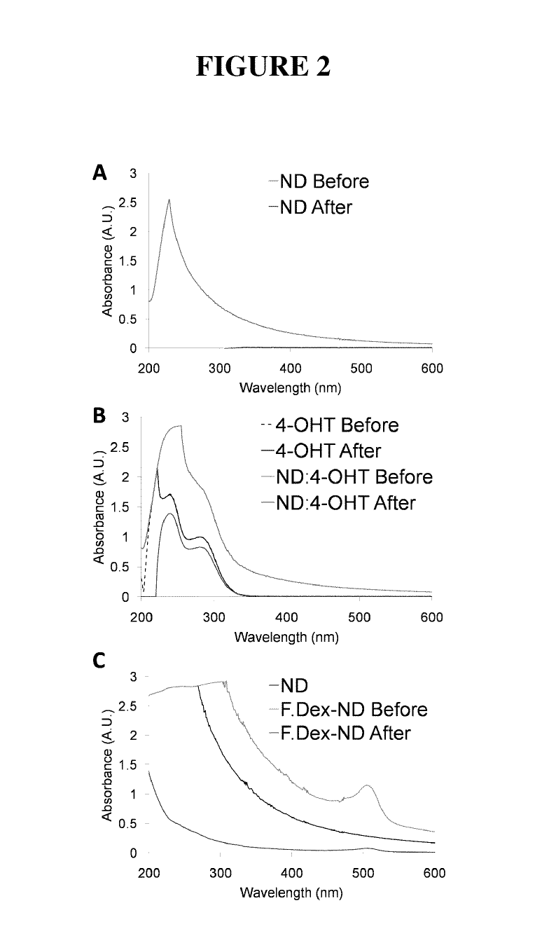

ND:4-OHT interfacing was further confirmed quantitatively via ND pulldown assays coupled with UV-Vis spectrophotometric analysis (FIG. 2). A wavelength scan of uncomplexed NDs revealed that the great majority of NDs pelleted upon centrifugation, leaving little ND remnants behind in the supernatant (FIG. 2A). In contrast, a similar control assay with uncomplexed 4-OHT, before and after centrifugation was performed. An insignificant change in the UV-Vis absorbance demonstrated that in the absence of NDs, the same amount of free 4-OHT resided within the supernatant despite centrifugation (FIG. 2B). This reading served as a control to mark the changes in uncomplexed 4-OHT dispersion due to centrifugation. Therefore, it logically follows that ND:4-OHT complexes would pellet upon centrifugation, and uncomplexed 4-OHT would remain in the supernatant resulting in a decrease in 4-OHT concentration in the supernatant. This conjugation scheme was tested by measuring UV-Vis absorption for ND:4-OHT solutions before and after centrifugation at the same conditions and concentrations as the ND and 4-OHT controls (FIG. 2B). This experiment revealed a marked change in absorbance between the uncentrifuged and centrifuged ND:4-OHT samples, which implied that a significant amount of 4-OHT was pulled down in conjunction with the NDs, possibly through ND:4-OHT physisorption and clustering. These data confirm the observation that NDs enhance the solubility of 4-OHT in 25% DMSO as compared to 4-OHT alone. The same clustering effect was observed in pulldown assays using FITC-labeled Dex-ND complexes (FIG. 2C).

While the present invention is not limited to any particular mechanism and an understanding of the mechanisms is not necessary to practice the invention, similar to the interaction between Purvalanol A and ND, the interplay between 4-OHT and the NDs is also thought to be mainly attributed to physisorption and/or electrostatic in nature. As a result of potential dipoles that exist from the structure of 4-OHT, the presence of surface carboxyl groups could have contributed to the interfacing between the two components in order to preserve ND:4-OHT sequestering.

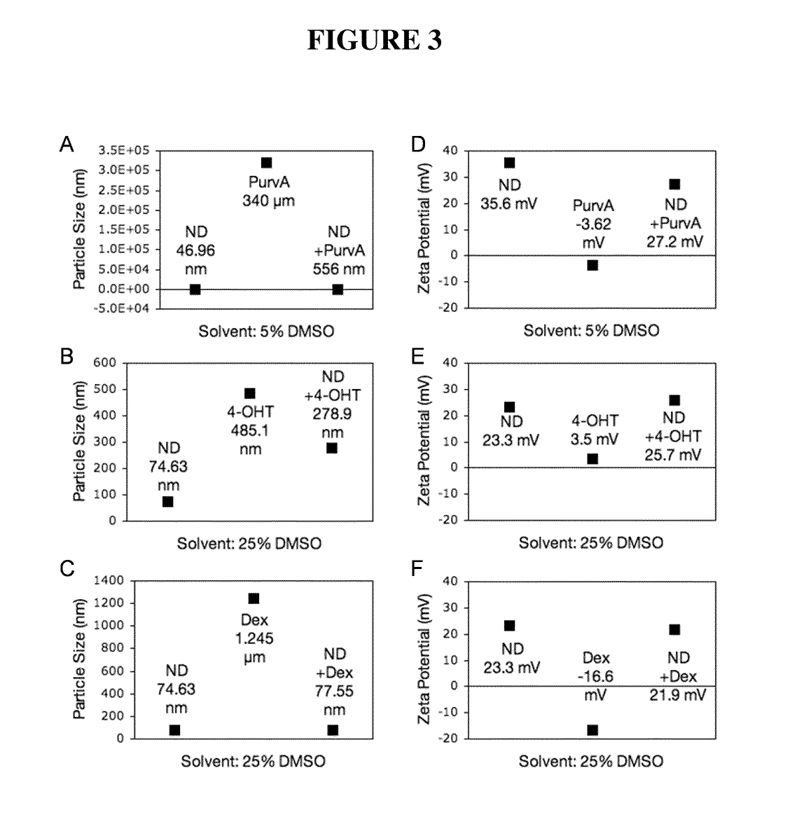

To determine the physical effects of the electrostatic interactions between NDs and respective therapeutics, the particle sizes and zeta potentials of the complexes were examined via dynamic light scattering (DLS) (FIG. 3). The lack of solubility of the three drugs is ultimately a result of particle aggregation upon titration with water from DMSO. In 5% DMSO, NDs had a mean diameter of 46.96 nm, and Purvalanol A, 4-OHT, and Dex aggregated into 340 μm 485.1 nm, and 1.245 μm particles, respectively. Upon complexing with NDs, the average Purvalanol A, 4-OHT, and Dex particle sizes decreased to 556 nm, 278.9 nm, and 77.55 nm, respectively (FIG. 3A-3C). The decrease in particle size for all drugs tested is evidence of physisorption of drug molecules to the surface of the ND particles. These data demonstrate the ability for drug molecules to associate with NDs and as a result, experience a significant decrease in particle size, in some cases by several orders of magnitude. Additionally, the zeta potentials of each drug were shown to become more positive upon association with NDs (FIGS. 3D-3F). This increased zeta potential would contribute to the increased solubility of ND-drug complexes in water due to water molecules having a greater affinity for forming hydration shells around charged complexes compared to neutral molecules.

Moreover, the increased drug solubility that has been demonstrated may also have potential clinical advantages pertaining to increased therapeutic efficacy as it has been shown that cellular internalization is enhanced when particles are both smaller and slightly positively charged [4]-42]. Both properties are favorable for internalization across the negatively charged plasma membrane and may facilitate drug uptake via endocytosis and pinocytosis.

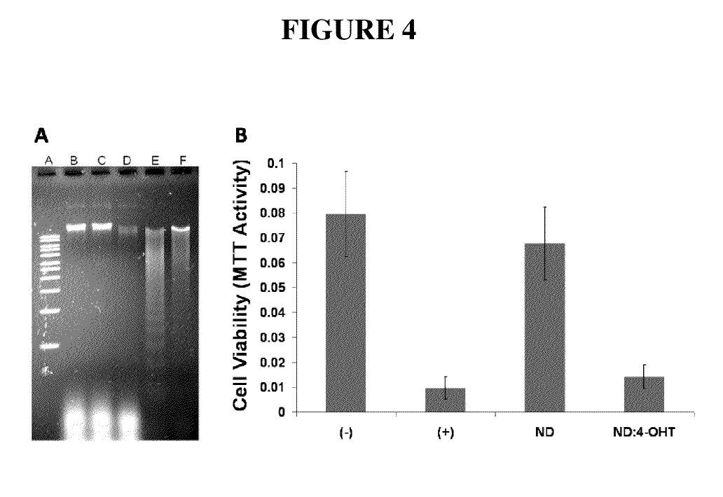

To assess drug functionality following enhanced dispersion in water via ND complexing, DNA laddering assays were performed to confirm Purvalanol A-induced DNA fragmentation (FIG. 4A). Fragmentation was evident in both ND:Purvalanol A and Purvalanol A samples, demonstrating the retained biological activity of Purvalanol after undergoing sequestration to and release from the NDs. As such, the assay attests to the capability of NDs not only to disperse a poorly water-soluble drug in an aqueous solution, but also to maintain Purvalanol A therapeutic activity.

Additionally, the chemotherapeutic effects of the ND:4-OHT complexes were evaluated via MTT cell viability assays (FIG. 4B). FIG. 4B shows no significant difference in cell viability between MCF-7 cultures with and without NDs, which further confirms the reported biocompatibility of NDs. Moreover, comparison of cell viability between ND:4-OHT complexes and the 4-OHT positive control demonstrates that the ND:4-OHT complexes have the same magnitude of chemotherapeutic potency as the drug alone. Exposure to the ND:4-OHT complexes decreased cell viability over seven-fold compared to the negative control and ND cultures. Most importantly, these observations collectively confirm the ability for NDs to increase 4-OHT dispersion in water via formation of a water-soluble ND:4-OHT complex, while maintaining drug functionality.

This Example has demonstrated the application of NDs towards enhancing water-dispersion of poorly water-soluble therapeutics. Purvalanol A and 4-OHT/Dexamethasone were selected as model drugs as they are characteristically soluble in DMSO and ethanol, respectively. Furthermore, due to the functionality of Purvalanol A as a broadly relevant cyclin dependent kinase inhibitor/chemotherapeutic and 4-OHT as a potent breast cancer drug, their enhanced solubility in water is catalytic towards their continued translation to the clinical realm. NDs represent a class of medically-significant nanomaterials that are capable of enabling rapid and high-throughput complex formation with hydrophobic drugs to enable their suspension in water and clinically-relevant applications. As such, NDs serve as scalable platforms that can facilitate facile delivery of these drugs with maintained biocompatibility.

### Example 2

### Alkaline-Sensitive Nanodiamond-Protein Complexes

This example describes the preparation and testing of nanodiamond-protein complexes.

### Cell Culture

The murine cell lines RAW 264.7 macrophages and 3T3-L1 fibroblasts (ATCC Manassas, Va.) were maintained in DMEM (Cellgro, Herndon, Va.) with 1% penicillin/streptomycin (Cambrex, East Rutherford, N.J.) containing 10% FBS (ATCC) and 10% CBS (ATCC), respectively, at 37° C. in 5% CO₂. 3T3-L1 fibroblasts were cultured in DMEM supplemented with 10% CBS until reaching 90% confluency, whereupon adipocyte differentiation commenced in accordance to previously established protocols [35, 36]. Media was replaced with DMEM, 10% FBS, 0.86 μM insulin, 0.25 μM dexamethasone and 0.5 mM isobutylmethylxanthine (IBMX) (Sigma Aldrich St. Louis, Mo.) for 4 days, renewing the media on day 2. Media was replaced on day 4 with DMEM, 10% FBS and 0.86 μM insulin, and again on day 6 with DMEM, 10% FBS for an additional 4 days. Cells were fully differentiated on day 10, and subsequently cultured in DMEM, 10% FBS and 1% penicillin/streptomycin.

### Formation of ND-Insulin Complex

Nanodiamonds (NanoCarbon Research Institute Co., Ltd., Nagano, Japan) dispersed in water underwent ultrasonication for 4 hours (100 W, VWR 150D Sonicator) to further disperse ND aggregates. Aqueous insulin was then added to ND solutions at varying ratios and mixed thoroughly to promote insulin binding to the NDs by physical adsorption.

### Protein Characterization

FITC-labeled insulin (Sigma-Aldrich) was dissolved in a 1 mM stock solution. Samples were measured using a Beckman Coulter DU730 UV/vis spectrophotometer (Fullerton, Calif.) at peak absorbance of approximately 494 nm (peak varied with solvent). Bovine insulin (Sigma-Aldrich), dissolved in acetic acid (pH 3) and neutralized with 1 mM NaOH, was used to supplement the results from FITC insulin. Protein detection was performed using the Micro BCA Protein Assay Kit (Thermo Scientific, Rockford, Ill.), measuring absorbance at 562 nm.

### FT-IR and TEM Characterization

A 4:1 ratio of NDs to insulin was prepared, centrifuged at 14,000 rpm for 2 hours and the supernatant removed. The ND-insulin pellet was rinsed with water and dried under vacuum. Individual ND and insulin samples were also prepared by dehydrating each respective solution. Additionally, a sample of NaOH-treated ND-insulin was made for TEM imaging by adding 1 mM NaOH adjusted to pH 10.5 to ND-insulin, centrifuging for 2 hours at 14,000 rpm and isolating the ND pellet. Samples were characterized at room temperature using a Thermo Nicolet Nexus 870 FT-IR spectrometer and a Hitachi H-8100 TEM (Pleasanton, Calif.).

### DLS Analysis

Hydrodynamic size and zeta potential of samples was measured with a Zetasizer Nano (Malvern Instruments, Worcestershire, United Kingdom). NDs and insulin were prepared as previously described. Briefly, the particles were suspended in buffer with corresponding pH at a concentration of 50 mg/mL. The size measurements were performed at 25° C. and at a 173° scattering angle. The mean hydrodynamic diameter was determined by cumulative analysis. The zeta potential determinations were based on electrophoretic mobility of the microparticles in the aqueous medium, which was performed using folded capillary cells in automatic mode.

### Insulin Adsorption and Desorption

Determination of insulin adsorption to NDs was performed by protein detection assays before and after centrifugation. Insulin was added to a ND suspension, centrifuged at 14,000 rpm for 2 hours and the resultant solution extracted and quantified. Detection of desorbed insulin was performed by adding alkaline solutions of 1 mM NaOH, adjusted for varying pH, to samples of ND-insulin in suspension. Binding ratios were determined similar to the adsorption test.

Additionally, a 5-day desorption test was conducted to determine cumulative insulin release. Samples were prepared by combining NDs and insulin (4:1 ratio), centrifuging at 14,000 rpm for 2 hours and extracting the remaining solution to remove any non-adsorbed insulin. Subsequently, a 1 mM NaOH solution adjusted to pH 10.5 was added to the samples, mixed thoroughly and centrifuged after a 24-hour period to determine protein concentration utilizing a BCA assay. In addition to alkaline-mediated release, water was added to a separate set of samples. The samples were replenished with NaOH or water after each measurement for the respective conditions, and the process was repeated every 24 hours over the course of 5 days.

### MTT Cell Viability Assay

RAW 264.7 murine macrophages were plated in 96-well plates, serum-starved for 8 hours and then incubated for 24 hours. Post-starvation media was composed of the following conditions: DMEM, 0.1 μM insulin, 1 μM insulin, DMEM 10% FBS, approximately 0.1 μM insulin released from ND-insulin complex by NaOH at pH 10.5 (insulin present in media), resultant solution from centrifuged ND-insulin in water, ND-insulin treated with NaOH at pH 10.5 (1 μM total insulin, ND-insulin complex present in media) and ND-insulin (1 μM total insulin, ND-insulin complex present in media). Insulin released from NDs was prepared by centrifuging samples of NDs with adsorbed insulin in NaOH and extracting the resultant solution, which could be reconstituted with media to 0.1 μM insulin. In a similar fashion, water was utilized as a neutral solution for relevant desorption analysis. Methylthiazolyldiphenyl-tetrazolium bromide (MTT) solution (Sigma-Aldrich) was added corresponding to 10% of total volume, and then incubated for 3 hours. After formazan crystal formation, the media was removed and MTT solvent, 0.1 N HCl in anhydrous isopropanol (Sigma-Aldrich), was added to samples to solubilize the MTT dye. Sample absorbance measurements occurred at 570 nm, accounting for background at a wavelength of 690 nm.

### Quantitative RT-PCR

RT-PCR procedures were conducted as described previously [35]. 3T3-L1 adipocytes were plated in 6-well plates, serum-starved for 4 hours and then recovered in media solutions of DMEM, 0.1 μM insulin, approximately 0.1 μM insulin released from ND-insulin by NaOH (pH 10.5), resultant solution from centrifuged ND-insulin in pH-neutral water, ND-insulin treated with NaOH (1 μM total insulin) and NDs with bound insulin (ND-insulin, 1 μM total insulin). Preparations of media solutions containing DMEM, insulin, NDs and NaOH were conducted in a similar fashion to those implemented for the MTT assay. RNA isolation was completed by lysing cells with TRIzol reagent (Invitrogen Corporation, Carlsbad, Calif.) and added to chloroform to obtain genetic material by centrifugation. cDNA synthesis was performed using the iScript Select cDNA Synthesis Kit (Bio-Rad, Hercules, Calif.). PCR expression of the Ins1 and Csf3/G-csf genes (Integrated DNA Technologies, Coralville, Iowa) were quantified by the MyiQ Single Color Real-Time PCR machine (Bio-Rad, Hercules, Calif.) using SYBER Green detection reagents (Quanta Biosciences, Gaithersburg, Md.). The Rp132 gene (Integrated DNA Technologies) served as the housekeeping gene for normalization of cDNA among samples. The primer sequences for genes are given: Ins1, 5′-AGGTGGCCCGGCAGAAG-3′ (SEQ ID NO:1) and 5′-GCCTTAGTTGCAGTAGTTCTCCAGCT-3′ (SEQ ID NO:2); Csf3/G-csf, 5′-CCAGAGGCGCATGAAGCTAAT-3′ (SEQ ID NO:3) and 5′-CGGCCTCTCGTCCTGACCAT-3′ (SEQ ID NO:4); Rp132, 5′-AACCGAAAAGCCATTGTAGAAA-3′ (SEQ ID NO:5) and 5′-CCTGGCGTTGGGATTGG-3′ (SEQ ID NO:6).

### FT-IR and TEM

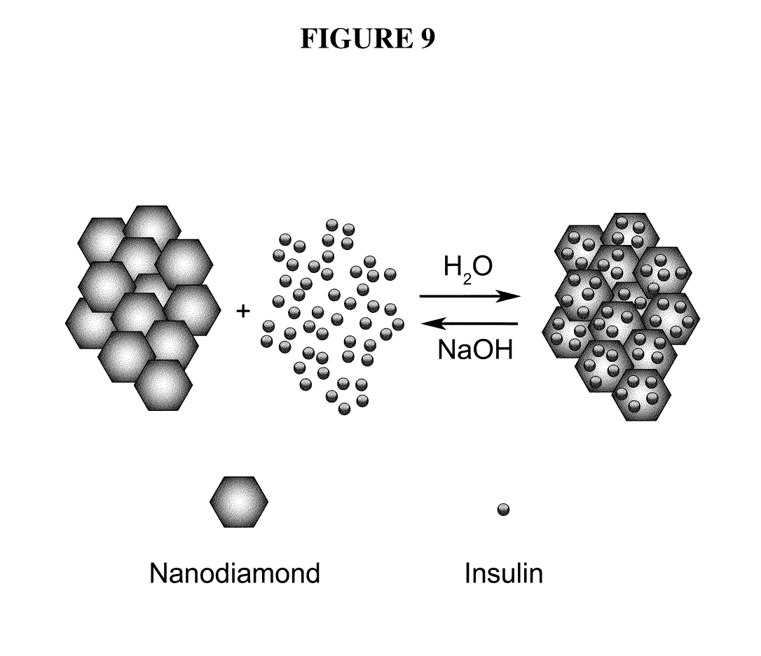

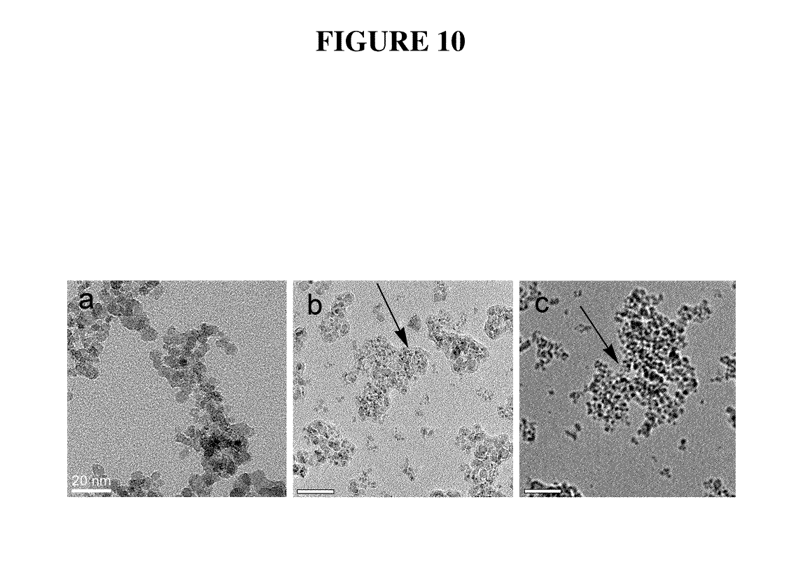

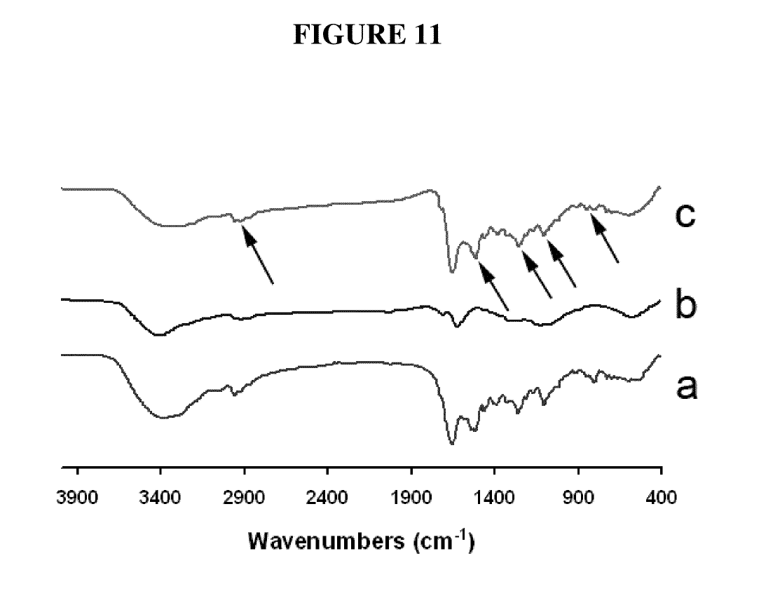

While the present invention is not limited to any particular mechanism and an understanding of the mechanism is not necessary to practice the present invention, illustrated in FIG. 9 is a representation of the proposed mechanism of insulin adsorption and desorption in neutral and alkaline solutions, respectively. Transmission electron microscopy (TEM) images in FIG. 10 show bare NDs (a), NDs with adsorbed insulin (b) and the ND-insulin complex after treatment with NaOH (c). In (b) there is an apparent layer of material coating the NDs approximately 5-10 nm in thickness. The NaOH-treated ND-insulin sample (c) qualitatively shows a diminished layer of material on the NDs, suggesting NaOH treatment of ND-insulin removed the material present on the ND surface. Fourier transform infrared (FT-IR) spectroscopy (FIG. 11) suggests the presence of insulin on NDs. Samples of insulin (1), bare NDs (2) and ND-insulin (3) are shown, with spectra peaks on ND-insulin indicating characteristic peaks similar to insulin.

### DLS Analysis

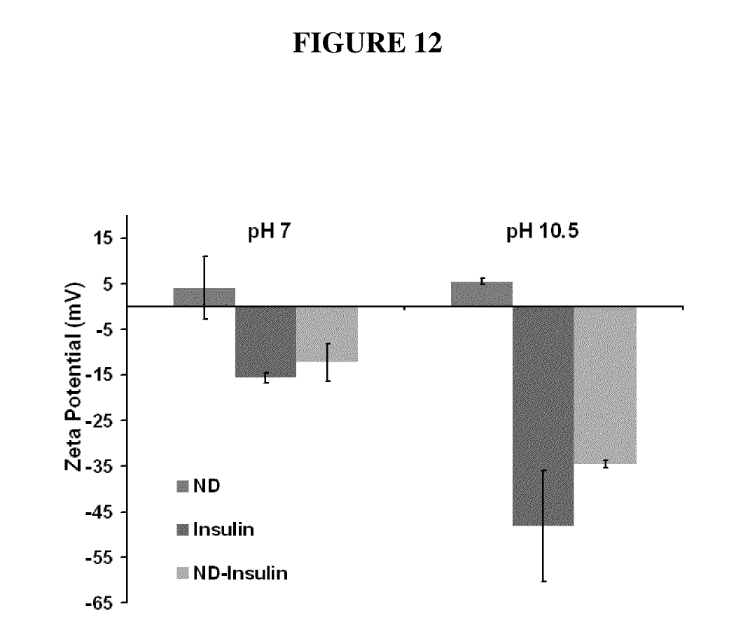

The interaction between NDs and insulin was characterized by means of dynamic light scattering (DLS) analysis, revealing hydrodynamic nanoparticle cluster size and polydispersity index summarized in Table 1 and zeta potential illustrated in FIG. 12. Average ND cluster size remained similar at pH 7 and 10.5, whereas insulin showed a larger average size at pH 10.5. The ND-insulin complex demonstrated an average size comparable to bare NDs and a decreased polydispersity index. NDs exhibited a slightly positive zeta potential at both pH 7 and 10.5, while insulin and ND-insulin resulted in negative values. The zeta potential of insulin and ND-insulin at pH 10.5 was substantially more negative than similar samples at pH 7.

### Adsorption

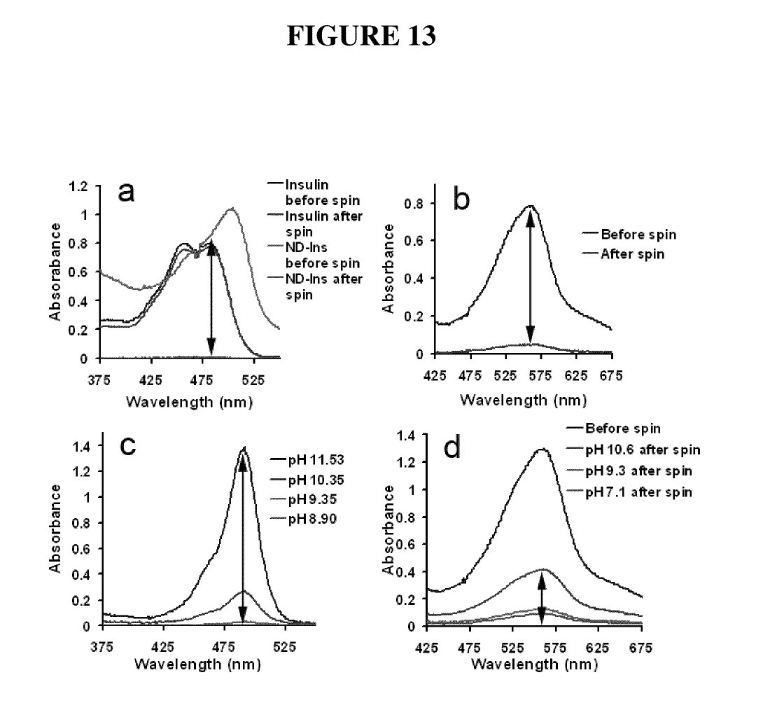

FIG. 9 shows a hypothetical schematic of how insulin in neutral solutions will bind by physical adsorption to NDs. FITC insulin samples of varying concentrations were mixed thoroughly with 100 μg/mL NDs to promote adsorption. Absorbance spectra for ND-insulin (FIG. 13-a) differ from that of aqueous insulin due to the adsorption of insulin to NDs. The ND-insulin complex, however, retains the spectral characteristics necessary to quantify the presence of insulin. The molecular weight of NDs, in addition to any adsorbed material, allows for the separation of components via centrifugation. Separation and analysis of remaining solutions yields supporting data concerning loading capacity and resultant release from NDs. FIG. 13-a illustrates protein adsorption of FITC insulin at a 5:1 ratio of NDs to insulin, demonstrating 89.8±8.5% binding in water. ND-insulin and insulin samples were measured before and after centrifugation, resulting in lower insulin concentrations of the ND-insulin sample as compared to the insulin sample due to centrifugation.

A similar test was conducted using standard bovine insulin implementing the BCA protein assay. Adsorption of 25 μg/mL insulin to 100 μg/mL NDs (4:1 ratio of NDs to insulin) demonstrated 79.8±4.3% binding, taking into account the pull-down effect of centrifugation on insulin. FIG. 13-b shows the absorbance spectra for ND-insulin samples before and after centrifugation, with peak absorbance at 562 nm. The absorbance of the centrifuged sample is significantly lower than that of the initial sample.

Protein binding ratios were determined by calculating the difference in absorbance between initial and centrifuged samples, and subtracting the difference in initial and centrifuged insulin control. The insulin control must be taken into consideration due to the slight gradient formed when insulin is centrifuged.

### Desorption

The desorption assays were conducted in a similar manner as the adsorption assays. Aqueous solutions of FITC-labeled and standard insulin were added to ND suspensions at 5:1 and 4:1 ratios, respectively. Initial and centrifuged samples were measured, and the amount of insulin desorbed was calculated. Comparing released FITC insulin at pH values of 8.90, 9.35, 10.35 and 11.53, maximum desorption was demonstrated at the most alkaline pH (FIG. 13-c). Separate tests at pH 10.7 show the ND-insulin complex achieving 53.3±1.2% desorption. Standard insulin release from NDs at pH 7.1, 9.3 and 10.6 also showed the greatest elution occurred at a pH of 10.6 (FIG. 13-d). This desorption profile shows that insulin release demonstrates proportionality to the pH of solution. Separate tests conducted with NDs and insulin at a 4:1 ratio in the presence of NaOH at pH 10.5 resulted in a 31.3±1.6% release of insulin.

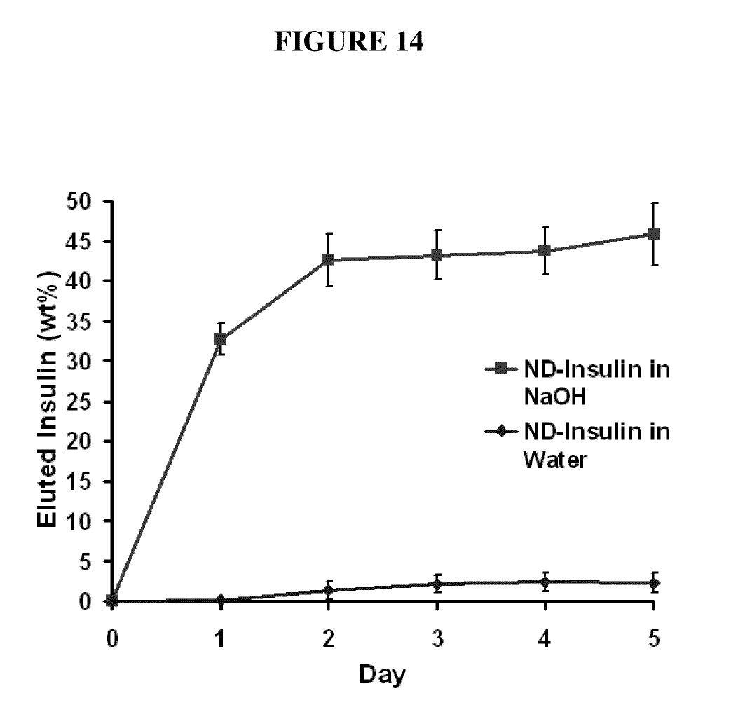

FIG. 14 illustrates the release of insulin from NDs over a period of 5 days in NaOH (pH 10.5) and water. Cumulative insulin eluted was quantified by weight percentage of total adsorbed insulin. The amount of insulin released by day 1 from alkaline conditions (pH 10.5) was 32.7±1.9 wt % compared to that of the water sample of 0.2±0.1 wt %, revealing a considerable difference in release between the two samples. By day 3 both samples tended to plateau and release significantly less insulin, and by day 5 the total amount of insulin eluted by NaOH and water was 45.8±3.8 wt % and 2.2±1.2 wt %, respectively. These values denote more than 20 times the amount of insulin released from the samples containing NaOH than those containing water.

### MTT Cell Viability Assay

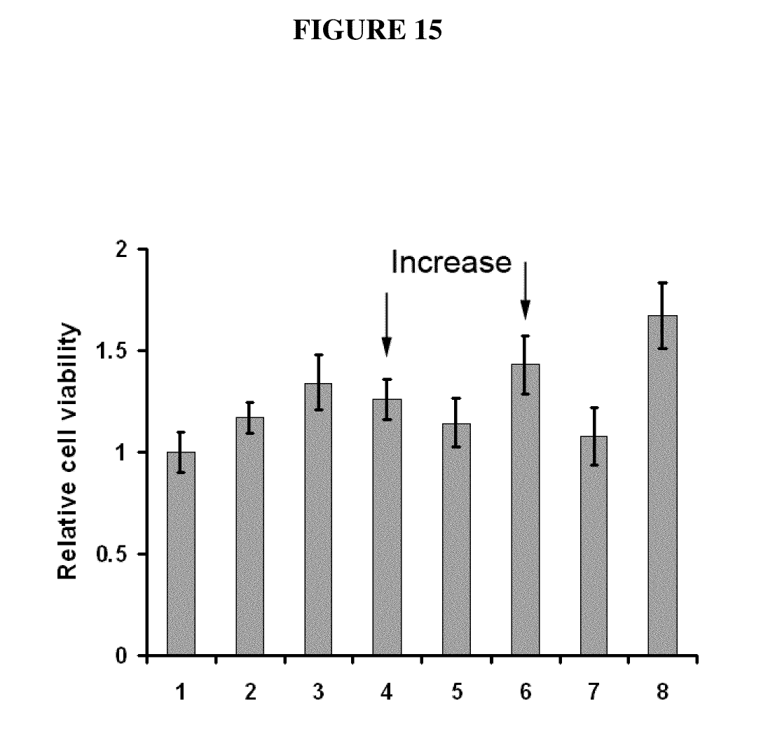

Cell viability tests under different insulin and ND conditions were performed (FIG. 15): DMEM (1), 0.1 μM insulin (2), 1 μM insulin (3), approximately 0.1 μM insulin released from ND-insulin complex by NaOH (pH 10.5) (4), resultant solution from centrifuged ND-insulin in water (5), ND-insulin treated with NaOH (1 μM total insulin) (6), NDs with bound insulin (ND-insulin, 1 μM total insulin) (7) and DMEM 10% FBS (8). Note that the amount of insulin adsorbed to NDs for both ND-containing samples is equal to 1 μM in media if insulin completely dissociates from the ND surface. Significantly higher relative viability occurred from 0.1 μM (2) to 1 1 μM (3) insulin, inferring increased viability at higher insulin concentrations. Relative viability for insulin released from ND-insulin by NaOH (4) is comparable to a relative viability between that of 0.1 μM to 1 μM insulin. Insulin desorbed by water (5) showed relative viability similar to that of 0.1 μM insulin, despite previous desorption results revealing insignificant levels of insulin in the resultant solution. ND-insulin treated with NaOH (6) demonstrated improved relative viability, greater than that of 1 μM insulin but less than 10% FBS media. ND-insulin (7) resulted in low relative cell viability comparable to DMEM and insulin released by water. For the ND-insulin treated with NaOH and ND-insulin conditions, NDs were present in the media during the recovery period allowing for cellular interactions with the NDs as compared to similar samples absent of NDs. Regular culture media, 10% FBS in DMEM (8), reflected the highest relative viability. An analysis of variance (ANOVA) statistical test was conducted yielding P<0.01, indicating a significant difference among sample groups.

### Quantitative RT-PCR

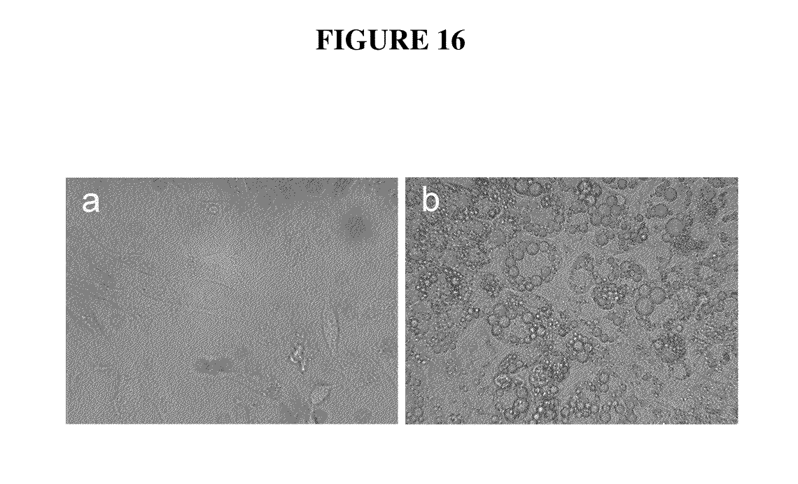

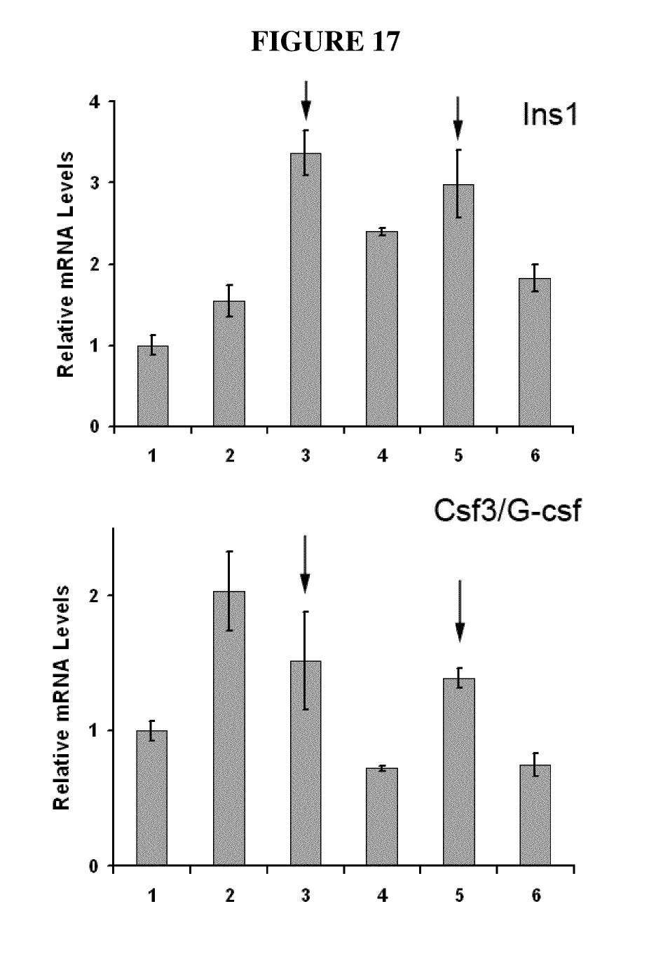

Pre-adipocyte differentiation yielded adipocytes by day 10 post-induction based on observations of morphology change and lipid vesicle formation in >90% of cells (FIG. 16). Pre-adipocytes (a) differ from adipocytes (b) by the clearly visible lipid vesicles. The effect of released insulin on adipocytes was quantified by RT-PCR for the genes Insulin 1 (Ins1) and Granulocyte colony-stimulating factor (Csf3/G-csf), and normalized to the housekeeping gene Ribosomal protein L32 (Rp132). The relative expression of Ins1 in response to varying media solutions is shown (FIG. 17-a). Compared to DMEM (1), insulin released by NaOH (3) and ND-insulin treated with NaOH (5) showed the highest relative expressions, indicating these conditions had the greatest effect on Ins1. Insulin released by water (4) and ND-insulin (6) resulted in moderate expression levels compared to the insulin-only condition (2) showing the lowest expression of Ins1. Csf3/G-csf relative expression is displayed in FIG. 17-b and shows a similar trend as with Ins1 in that both insulin released by NaOH (3) and ND-insulin treated with NaOH (5) demonstrate high expression levels, while insulin released by water (4) and NDs with bound insulin (6) are significantly lower. The effect of 0.1 μM insulin on Csf3/G-csf, however, was comparably higher than that of Ins 1. The ANOVA statistical test gave P<0.01, indicating a significant difference among sample groups.

### Physical Adsorption

Conditions during ND synthesis result in a heavily functionalized hydrophilic carbon surface of hydroxyl and carboxyl groups, which can lead to a characteristic surface charge in aqueous solutions [8, 28, 29]. Such functional groups present favorable conditions for the physical adsorption of proteins via electrostatic attraction between anionic end groups (—COO⁻) and protonated amino groups (—NH₃⁺) of polypeptides. In addition to charge-charge interactions, hydrogen bonds can form between —NH₃⁺ and —COO⁻ or other CO-containing surface groups, with H-bond binding energies between 10-30 kcal/mol [33, 34, 37]. Charged amino acid residues on the exterior of the insulin molecule contribute to its hydrophilicity and can be attracted to the ND surface. Although the isoelectric point of insulin is approximately 5.6[38], indicating a slightly negative net charge at neutral pH, the electrostatic interactions and H-bonding between ND functional groups and amine biomolecules may lead to attractive interactions. FIG. 9 illustrates, hypothetically, this concept of insulin adsorption to NDs in a neutral environment.

TEM imagery shows ND s after immersion in aqueous insulin (FIG. 10-b) with a visible layer of material coating the ND surface, as compared to bare NDs (a). Since the addition of insulin (b) is the only discriminating factor, it lends precedence to the material layer (thickness 5-10 nm) being identified as adsorbed insulin. The ND clusters seen in FIG. 10 boast very high surface area allowing for substantial insulin adsorption to functional groups on the NDs. In fact, ND characterization has previously demonstrated a remarkable surface area of 450 m²/g [9]. TEM imaging provides visual recognition of protein binding, and adsorption can be quantified by FT-IR spectroscopy. Insulin adsorption to NDs is validated by FT-IR characterization of insulin, bare NDs and NDs with bound insulin (FIG. 11). The characteristic spectra of insulin (a) is distinctly seen in the spectra of NDs with bound insulin (c), quantifiable results that otherwise would not be obtained from NDs without adsorbed insulin (b). TEM and FT-IR provide additional evidence of insulin adsorption to the ND surface.

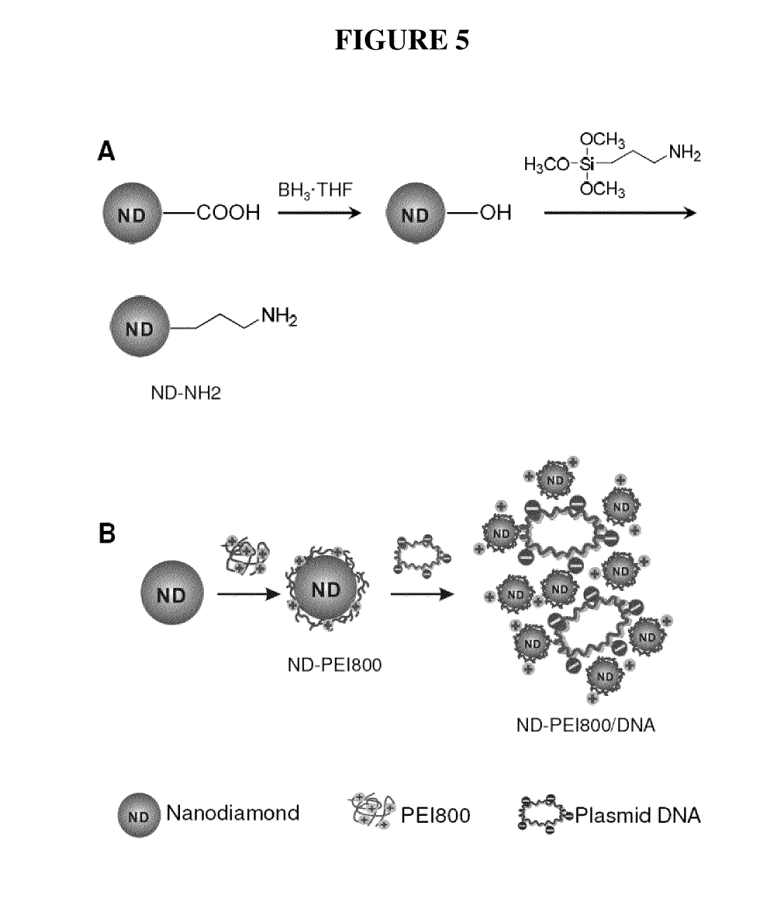

Further substantiation of the ND-insulin complex is given by UV/vis analysis. Adsorption tests revealed a 5:1 ratio of NDs to FITC insulin at optimal binding capacity (absence of excess insulin in resultant solution), demonstrating 89.8±8.5% adsorption. Absence of measurable absorbance of the centrifuged ND-insulin sample (FIG. 13-a) signifies considerable FITC insulin adsorption to NDs. The absorbance difference at 485 nm between initial and centrifuged ND-insulin samples is attributed to the molecular weight of NDs and settling of NDs with bound insulin during centrifugation, leaving trivial amounts of residual insulin in solution. A slight difference between initial and centrifuged insulin control samples is used to normalize adsorption values since the molecular weight of insulin compared to the aqueous solution allows for the separation of components. FIG. 5-a reveals altered absorbance spectra of ND-insulin when compared to that of insulin, with absorbance peaks of insulin and ND-insulin shifting from 485 nm to 505 nm. This peak shift is possibly due to a change in optical properties of the FITC molecule when FITC-labeled insulin adsorbs to NDs, indicating a possible conformational change in protein structure often observed in protein adsorption [39].

Similar results were obtained from standard bovine insulin adsorption tests with an optimal ND-to-insulin binding ratio of 4:1. A higher adsorption ratio for standard bovine insulin is expected given that the molecular weight of insulin as compared to that of FITC-labeled insulin. FIG. 13-b depicts BCA protein assay absorbance revealing contrasting peaks for initial and centrifuged ND-insulin samples associating to a substantial 79.8±4.3% insulin adsorption.

Insulin adsorption tests involving FITC-labeled and standard insulin are consistent with previous investigation verifying protein-ND binding [34] and exhibit exceptional adsorption capabilities, with approximately 80% of insulin binding to the ND surface at optimal ND-insulin ratios. The protein loading capacity of NDs as demonstrated by the adsorption tests imply a relatively efficient drug-loading process where the majority of available protein is adsorbed to the ND surface. The simple method of physical adsorption in aqueous solutions is ideal for drug delivery preparation methods by eliminating complex conjugation protocols that can affect the properties of the drug or substrate.

The physical interaction between NDs and insulin was also characterized via dynamic light scattering (Table 1).

TABLE 1

| | | Average Size | |
|---|---|---|---|
| | pH | (μm) | PDI |
| Nanodiamond | 7 | 1.67 ± 0.64 | 0.41 ± 0.084 |
| | 10.5 | 1.63 ± 0.66 | 0.30 ± 0.13 |
| Insulin | 7 | 1.59 ± 0.038 | 0.97 ± 0.046 |
| | 10.5 | 2.28 ± 0.66 | 0.99 ± 0.011 |
| Nanodiamond- | 7 | 1.69 ± 0.37 | 0.23 ± 0.18 |
| Insulin | 10.5 | 1.05 ± 0.081 | 0.40 ± 0.12 |

Table 1 shows a DLS analysis of hydrodynamic nanoparticle cluster size and the associated polydispersity index (PDI) at pH7 and 10.5. NDs exhibited similar size and PDI at both pH conditions, while insulin at pH 10.5 tended to form larger particles with an increased PDI. Upon formation of the ND-insulin complex the PDI decreased, suggesting NDs mediate a relatively even distribution size of clusters.

NDs formed clusters of similar hydrodynamic size and distribution at pH 7 and 10.5 while insulin aggregated into larger sizes within alkaline solutions. Upon complexing with NDs, the polydispersity index is not only reduced, but the zeta potential of the clusters also altered to a negative value (FIG. 12). The reduction of PDI as seen with the formation of ND-insulin complexes, compared to that of insulin, indicates a ND-mediated development of a more uniform nanomaterial-protein complex. NDs originally maintained a slightly positive zeta potential within alkaline solutions while insulin inherently possessed a negative zeta potential that further decreased in alkaline solutions. This zeta potential was retained upon introduction with NDs, implying insulin adherence onto the ND surface. This result is further verified since the cluster's zeta potential at pH 10.5 lies within a narrow confined range of values. The clear difference in zeta potential between bare NDs and ND-insulin suggests an interaction between NDs and insulin.

pH-Mediated Desorption

Release of insulin from the ND-insulin complex was observed in alkaline sodium hydroxide solutions and can be explained by a change in charge characteristics affected by pH modification. Insulin in aqueous environments at a pH above the isoelectric point may carry a negative net surface charge owing to the charge alteration of the functional end groups. Subsequently, the negative charge can become stronger with increased alkalinity and affect charge interactions with other species. Thus, the effect of pH on desorption is rather straightforward. Insulin molecules bound to charged functional groups on the ND surface via electrostatic interactions and hydrogen bonding will begin to display altered charge characteristics as the aqueous environment shifts from neutral to alkaline, and therefore release from the NDs by electrostatic repulsion.

The amount of desorbed insulin seems to be proportional to the pH of solution, showing increased insulin release in alkaline solutions (FIG. 13c-d). Absorbance spectra of FITC-labeled insulin desorption (FIG. 13-c) represent an increase in desorption as the pH shifts from 8.90 to 11.53, and a similar pattern is expressed in FIG. 13-d with standard insulin. These results are consistent with the pH-dependent desorption premise mentioned previously. The 31.3±1.6% desorption of standard insulin in NaOH demonstrates insulin is capable of being adsorbed and subsequently released from the ND surface into an aqueous medium.

Many practical applications necessitate the release of a drug over time, and in order to quantify the time-release of insulin a 5-day desorption test was conducted with NDs with bound insulin in both NaOH and water. The disproportion between the two release curves in FIG. 14 exemplifies the difference in desorption ability of alkaline and neutral solutions. Alkaline-mediated desorption reached 45.8±3.8% by day 5 compared to only 2.2±1.2% in water. The bulk of desorption occurred by day 1 of the test, suggesting a burst release of insulin from NDs. Day 2, however, did produce a moderate insulin release. The time-dependency of insulin release enables the ND-insulin complex to slow release insulin upon exposure to alkaline environments.

### Preservation of Protein Function

Results discussed in the previous section establish a basis for pH-mediated insulin desorption, yet practical use of such a system relies on the retained function of the drug upon release from the ND surface. The data obtained from MTT viability assays and RT-PCR suggest insulin function is indeed preserved subsequent to desorption as noted by cell viability and gene expression. Furthermore, insulin sequestered on the ND surface seems to remain inactive to cellular pathways despite the presence of the ND-insulin complex.

Cell viability data (FIG. 15) reveal an increase in cellular recovery with insulin released from NDs by NaOH and ND-insulin complex treated with NaOH, the latter comprised of NDs and desorbed insulin in media solution. The increased viability levels of cells in these two media conditions, as compared to DMEM baseline, signify the released insulin is activating cellular recovery pathways following the starvation period. Also, viability of ND-insulin treated with NaOH indicates cellular recovery occurs in the presence of the released insulin and the subsequent NaOH-treated NDs which may or may not result in bare ND surfaces. Previous investigation implementing serum-starvation and insulin recovery on RAW 264.7 macrophages [40] is consistent with the acquired MTT data showing insulin-mediated recovery.

Insulin released by water and ND-insulin, in contrast, yielded low viability levels, implying little or no insulin release in the neutral environment. The ND-insulin complex seems to prevent the adsorbed insulin from affecting cellular pathways even with insulin exposed on the ND surface. Proteins are often known to undergo a conformational change when adsorbed to a surface [39] leading to altered physical properties, and a change in the structure of insulin on the ND surface may prevent activation of cellular pathways. Effective isolation of insulin from a soluble environment until mediation by alkalinity is key to targeted insulin delivery of this system.

Gene expression from RT-PCR closely correlated with results from MTT viability assays. FIG. 17 shows relative expression of genes Ins1 and Csf3/G-csf, which are upregulated by insulin stimulation of adipocyte cells [35]. Expression levels for samples containing insulin released by NaOH and ND-insulin treated with NaOH increased for each gene, demonstrating the effectiveness of insulin after desorption from the ND surface. Absence of active insulin does not increase expression levels as noted by the DMEM baseline. Similar to the MTT results, insulin released by water and ND-insulin show reduced expression levels for each gene, indicating insufficient response to or reduced insulin concentration so as to activate cellular pathways. This suggests protein activity is retained for insulin desorbed from NDs as determined by genetic expression attributed to insulin stimulation. Additionally, adsorbed insulin, despite being bound to the ND surface, does not increase cell viability or gene expression. In this manner the ND-insulin complex presents a unique approach for targeted insulin (or other protein) delivery in alkaline environments while remaining stable in neutral solutions.

These findings also indicate that insulin adsorption and elution from NDs is pH-dependent, an observation that can be scaled for therapeutic purposes. While the present invention is not limited to any particular mechanism and an understanding of the mechanism is not necessary to practice the present invention, insulin desorption is shown to increase in alkaline environments possibly by action of a change in surface charge of the protein, thereby decreasing the propensity of ND-to-insulin attraction. Exploiting this pH-mediated desorption mechanism may provide unique advantages for enhanced drug delivery methods. It is well understood that insulin accelerates wound healing by acting as a growth hormone [4]-45]. Furthermore, previous investigations have confirmed an increase in alkalinity of wound tissue due to bacterial colonization, sometimes as high as pH 10.5 [46, 47]. Considering these two observations the ND-insulin complex may be used as a useful therapeutic drug delivery system for the treatment of wound healing. Administration of NDs with adsorbed insulin may be able to shorten the healing process and decrease the incidence of infection by releasing insulin in alkaline wound areas. Systemic activation of insulin would be limited as the release of insulin would occur at the site of injury. As such, the present invention provides for a targeted insulin-release mechanism directed at injury wounds as a regenerative therapy using NDs as an insulin vehicle.

Experiments conducted during development of embodiments of the present invention demonstrated the efficient, non-covalent adsorption of insulin to NDs by means of simple physical adsorption and has investigated the pH-dependency of protein desorption. Exposure of the ND-insulin complex to alkaline environments mediates the interaction between NDs and insulin resulting in protein release. Imaging methods and adsorption/desorption assays reveal effective binding of insulin to NDs and significant insulin release under alkaline conditions. MTT and RT-PCR analysis indicate preserved function following desorption, while adsorbed insulin remained largely inactive.

### Example 3

### Nanodiamond-Drug Binding Assays

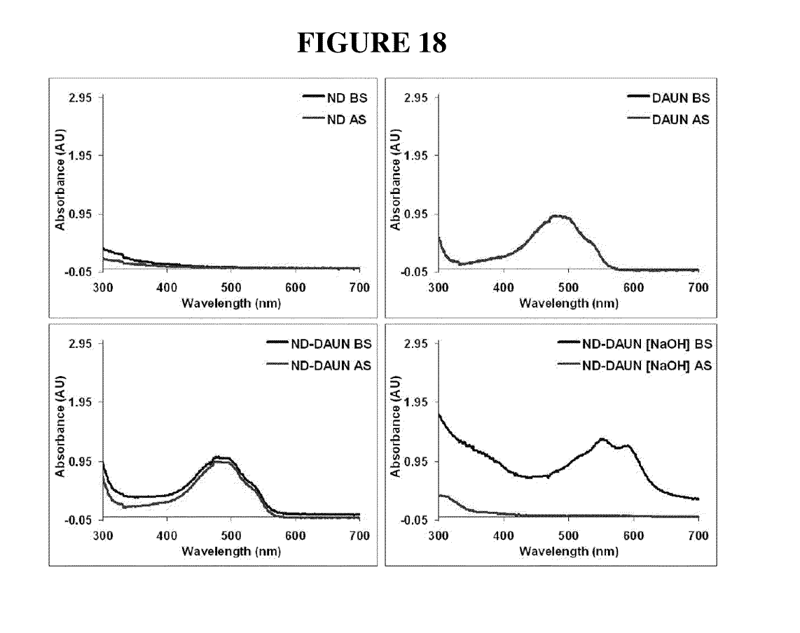

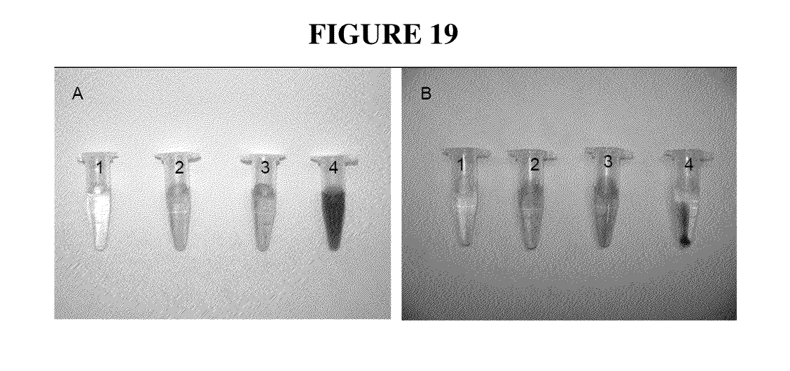

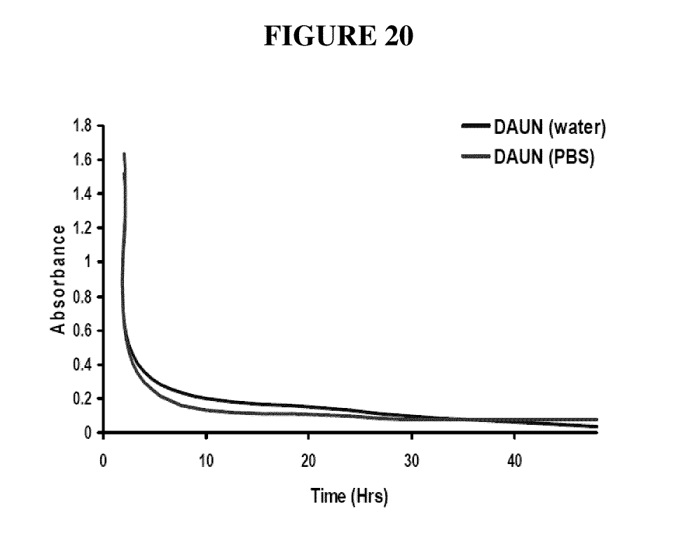

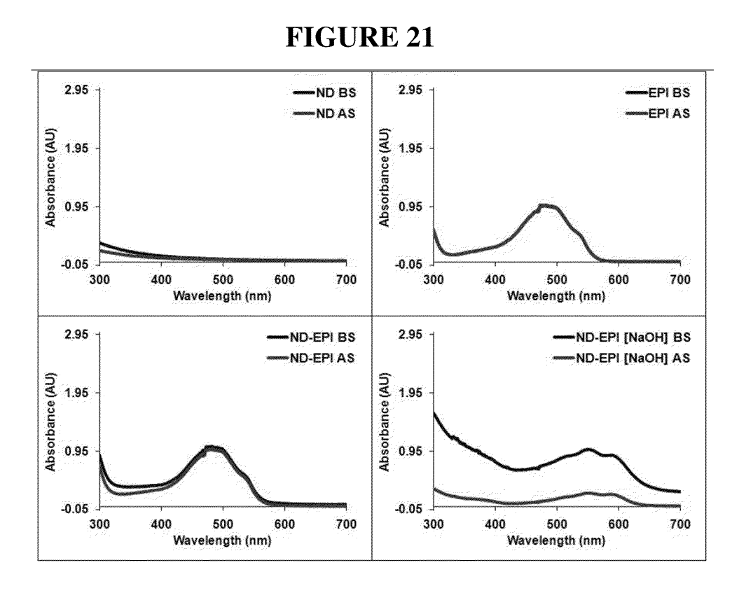

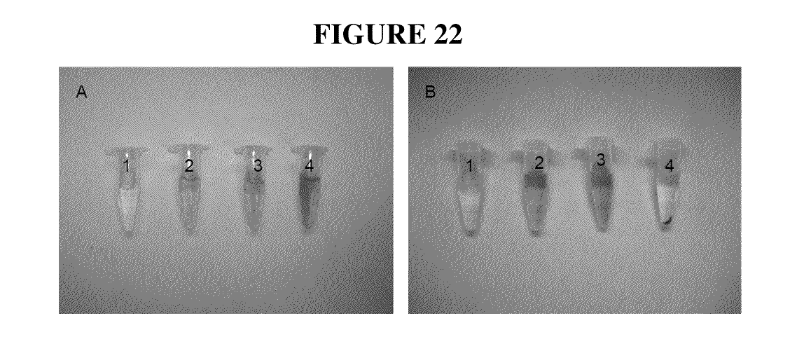

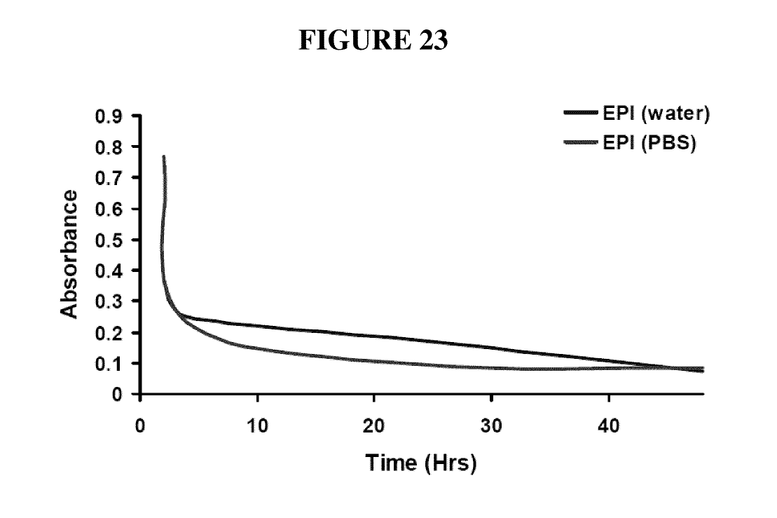

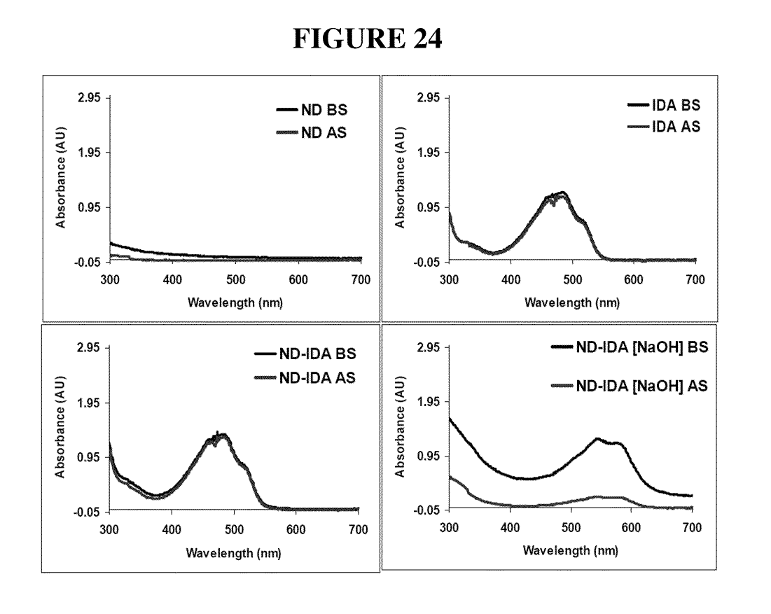

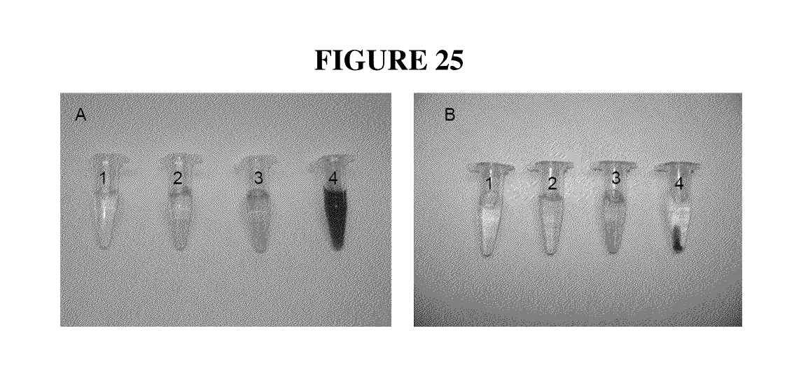

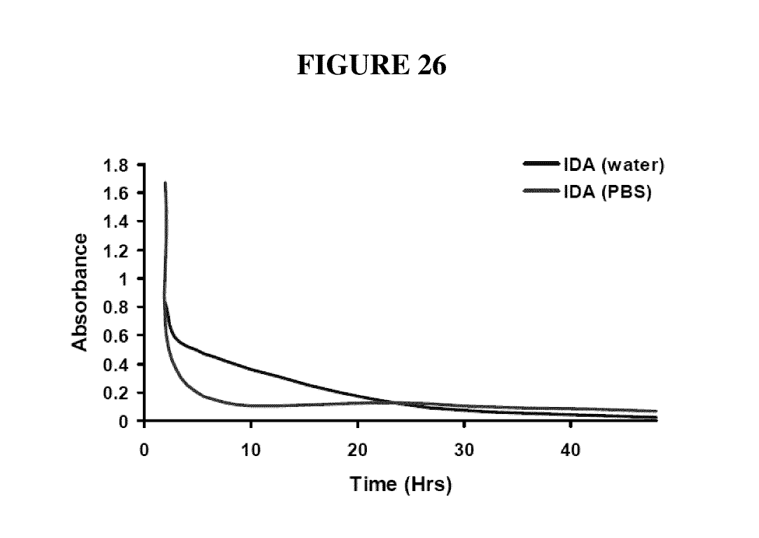

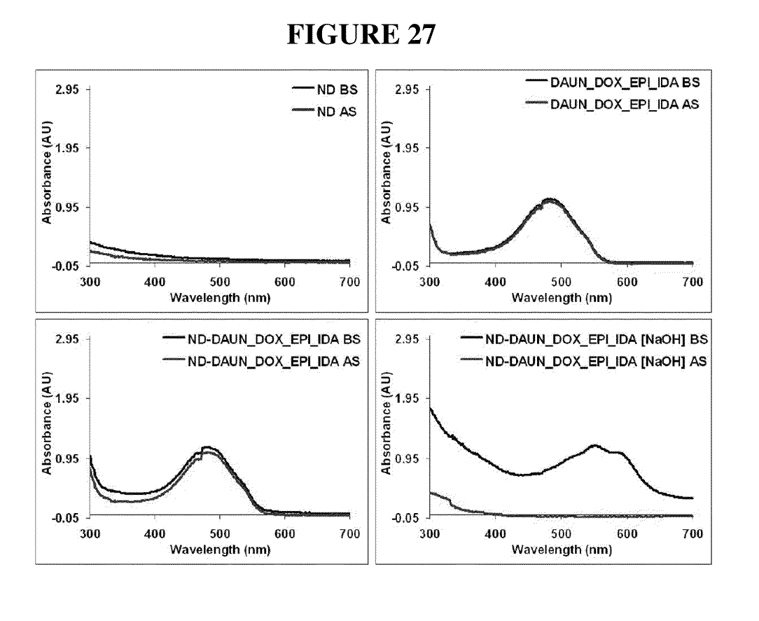

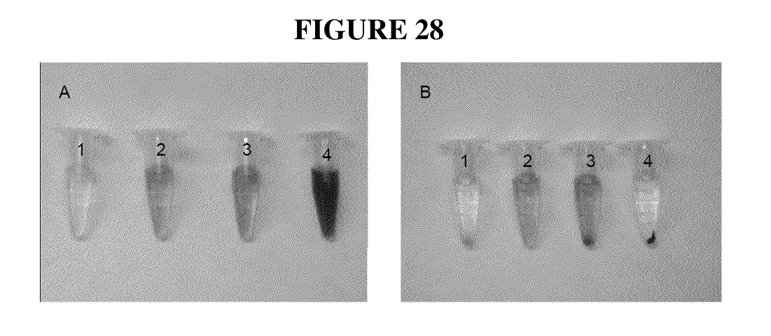

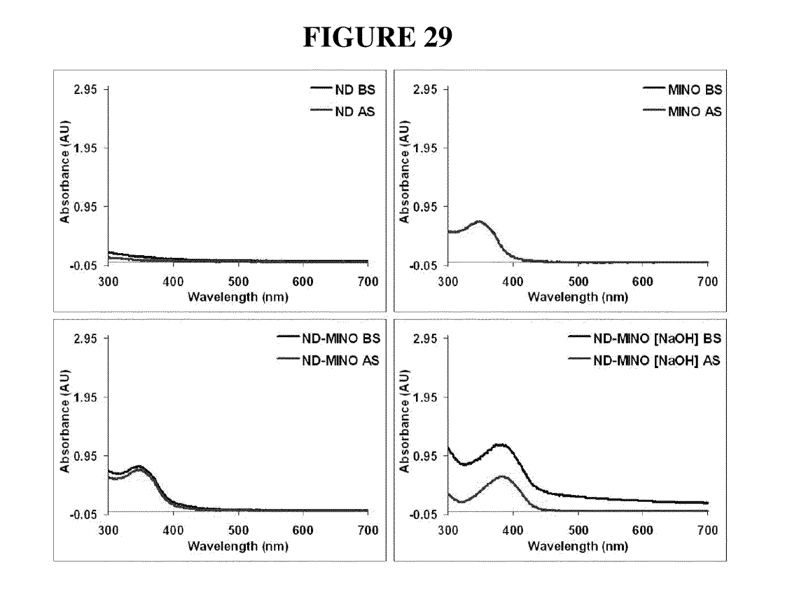

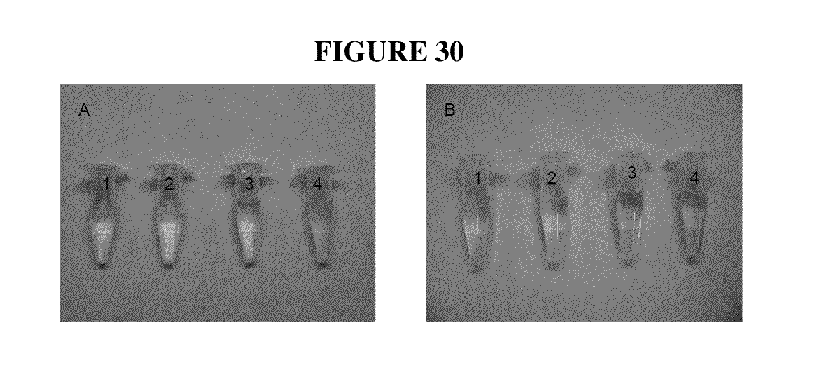

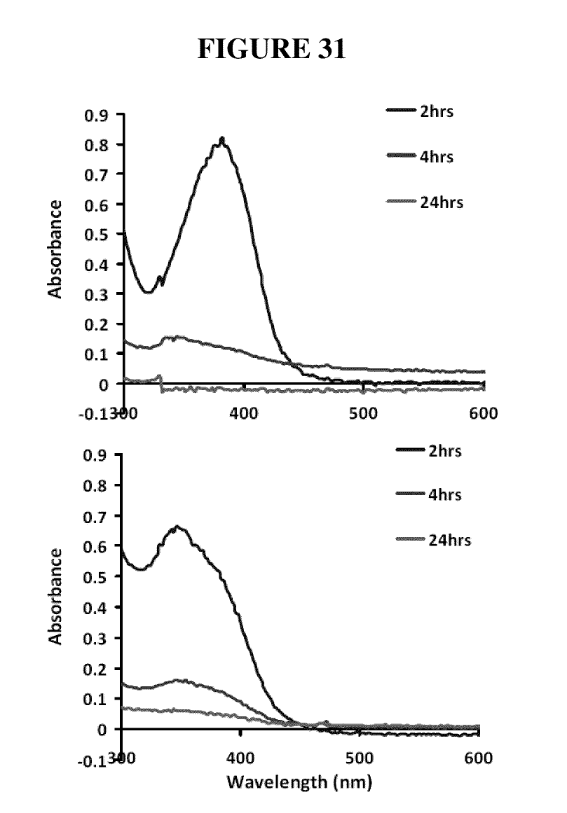

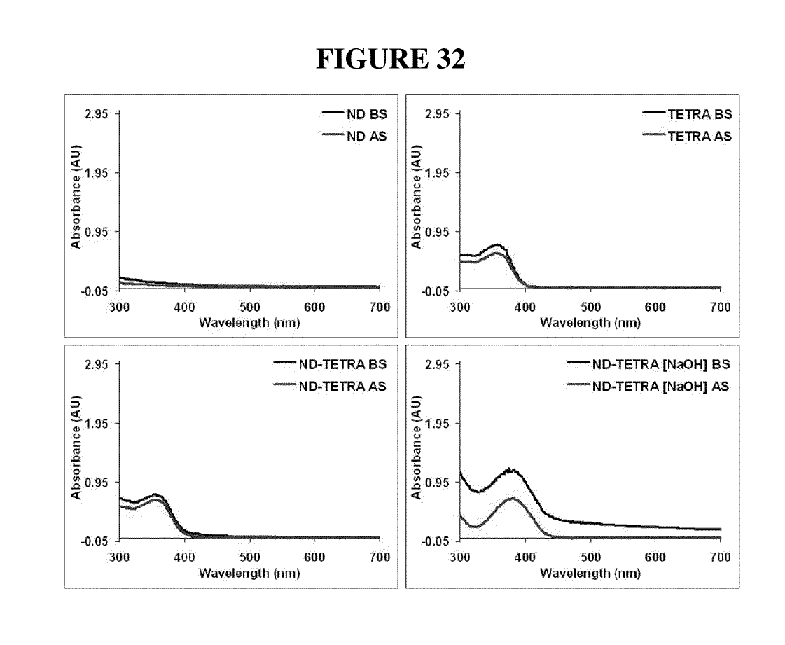

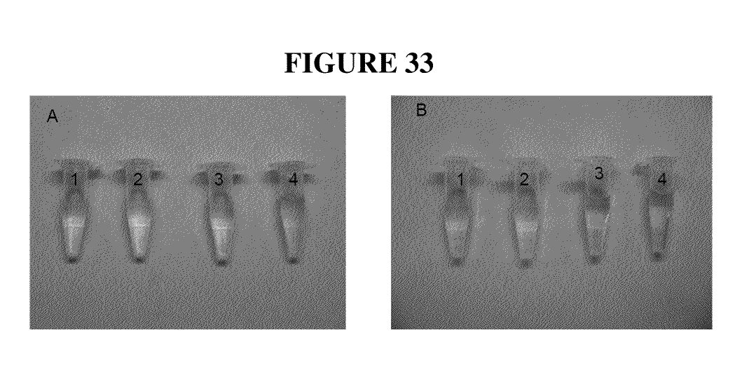

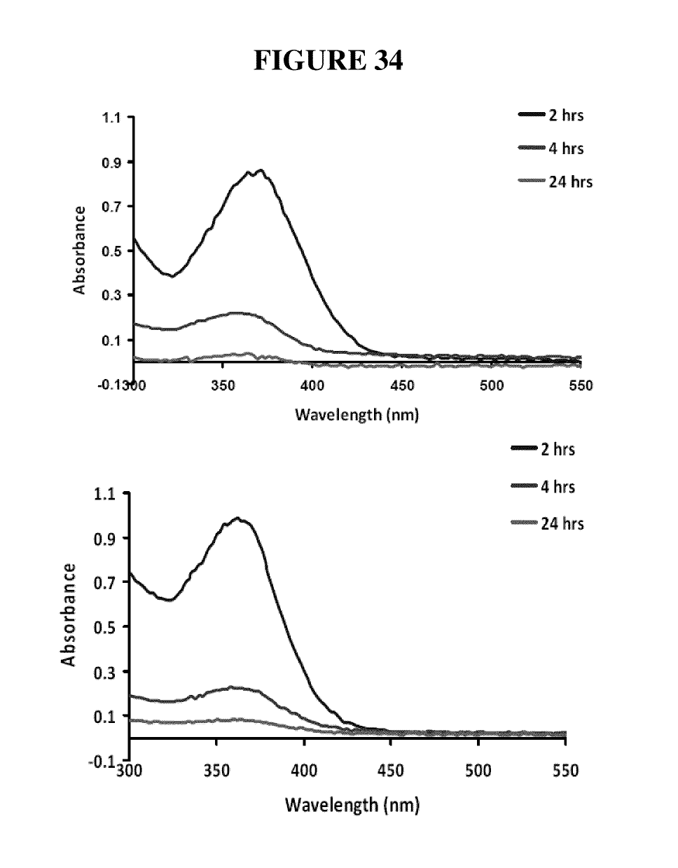

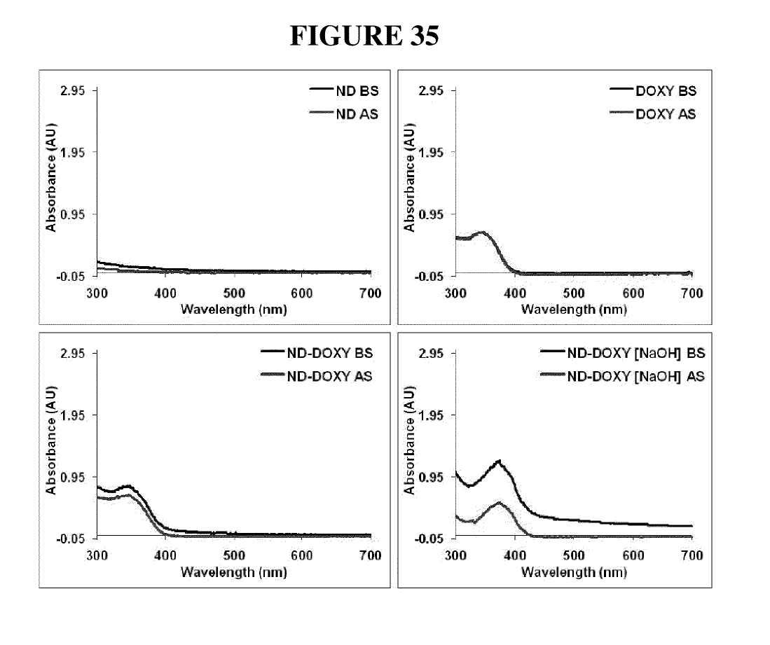

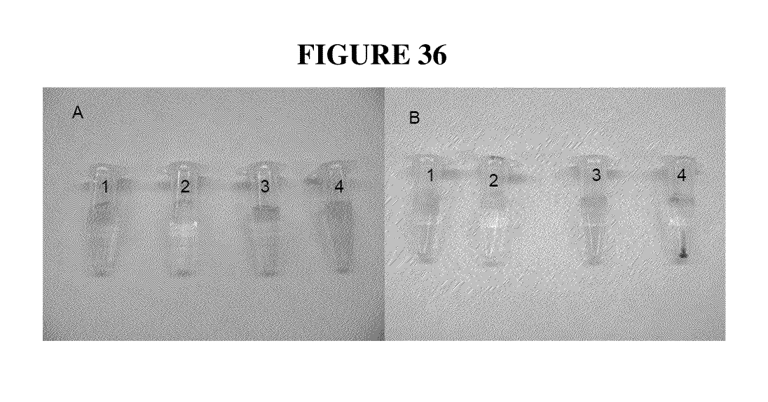

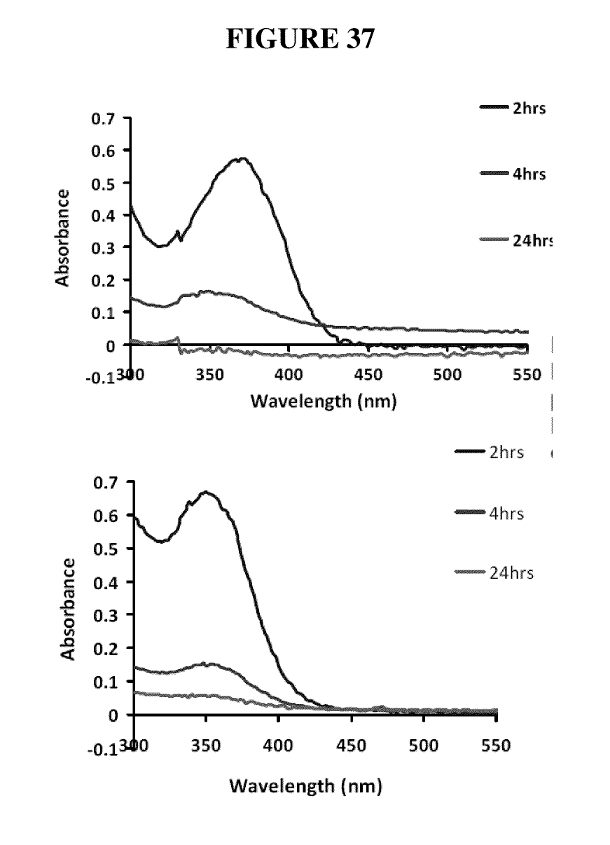

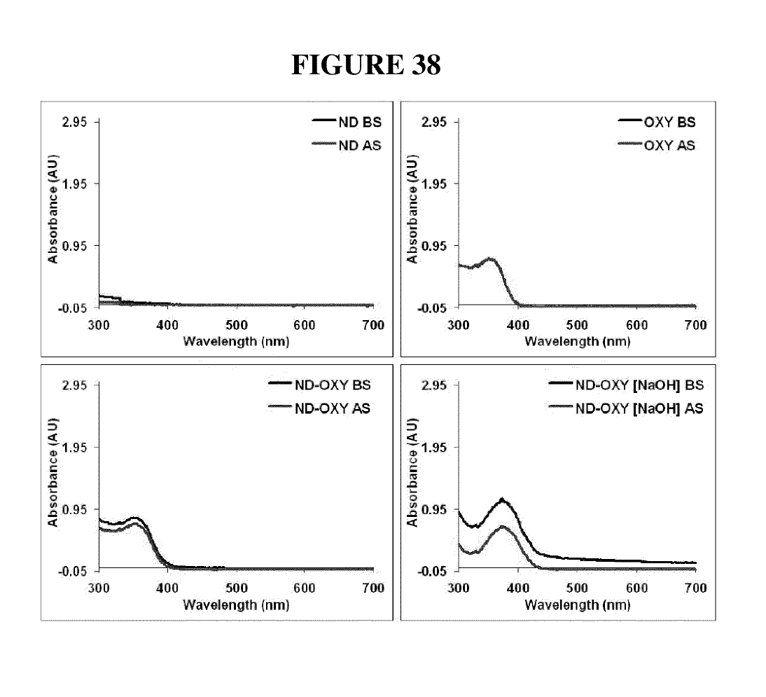

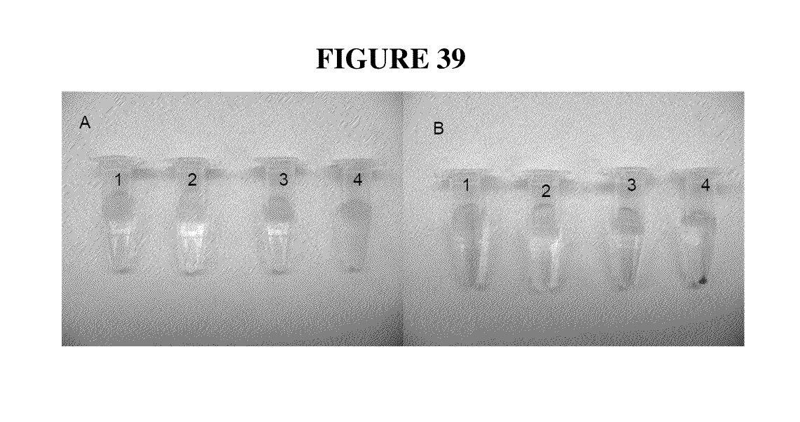

Nanodiamond-drug binding assays were performed during development of embodiments of the present invention to confirm the potent interaction between a broad array of anthracycline and tetracycline compounds. The binding efficiency of therapeutics such as daunorubicin, idarubicin, and others were analyzed using UV-vis spectrophotometry, as well as centrifugation assays (SEE FIGS. 18-39). In all cases, nanodiamond-drug interactions were capable of comprehensively pelleting the therapeutics (SEE FIGS. 19, 22, 25, 28, 30, 33, 36, and 39), indicating potent adsorption which was further confirmed via spectrophotometric analysis SEE FIGS. 17-18, 20-21, 23-24, 26-27, 29, SEE FIGS. 17-18, 20-21, 23-24, 26-27, 29,1-32, 34-35, and 37-38).

### REFERENCES

### References from Part I

### (Soluble Nanodiamond-Drug Complexes) and Example 1

1. Goga, A.; Yang, D.; Tward, A. D.; Morgan, D. O., Bishop, J. M. Inhibition of Cdk1 as a Potential Therapy for Tumors Over-Expressing MYC. Nat. Med. 2007, 13, 820-827.
2. Pantazis, P. Preclinical Studies of Water-Insoluble Camptothecin Congeners: Cytotoxicity, Development of Resistance, and Combination Treatments. Clin. Canc. Res. 1995, 1, 1235-1244.
3. Villerbu, N.; Gaben, A. M.; Redeuilh, G.; Mester, J. Cellular effects of purvalanol A: A specific inhibitor of cyclin-dependent kinase activities. Int. J. Canc. 2002, 97, 761-769.
4. May, F. E., Westley, B. R. Effects of Tamoxifen and 4-hydroxytamoxifen on the pNR-1 and pNR-2 Estrogen-Regulated RNAs in Human Breast Cancer Cells. J. Biol. Chem. 1987, 262, 15894-15899.
5. Rouanet, P.; Linares-Cruz, G.; Dravet, F.; Poujol, S.; Gourgou, S.; Simony-Lafontaine, J.; Grenier, J.; Kramar, A.; Girault, J.; Le Nestour, E.; Maudelonde, T. Neoadjuvant Percutaneous 4-Hydroxytamoxifen Decreases Breast Tumoral Cell Proliferation: A Prospective Controlled Randomized Study Comparing Three Doses of 4-Hydroxytamoxifen Gel to Oral Tamoxifen. J. Clin. Onco. 2005, 23, 2980-2987.
6. Kim, Y.; Dalhaimer, P.; Christian, D. A.; Discher, D. Polymeric Worm Micelles as Nano-Carriers for Drug Delivery. Nanotechnology 2005, 16, S484-S491.
7. Zhang, L.; Chan, J. M.; Gu, F. X.; Rhee, J.-W.; Wang, A. Z.; Radovic-Moreno, A. F.; Alexis, F.; Langer, R. S.; Farokhzad, O. C. Self-Assembled Lipid-Polymer Hybrid Nanoparticles: A Robust Drug Delivery Platform. ACS Nano 2008, 2, 1696-1702.
8. Zhang, L.; Radovic-Moreno, A. F.; Alexis, F.; Gu, F. X.; Basto, P. A.; Bagalkot, V.; Sangyong, J.; Langer, R. S.; Farokhzad, O. C. Co-Delivery of Hydrophobic and Hydrophilic Drugs from Nanoparticle-Aptamer Bioconjugates. ChemMedChem 2007, 2, 1268-1271.
9. Sheihet, L.; Dubin, R. A.; Devore, D.; Kohn, J. Hydrophobic Drug Delivery by Self-Assembling Triblock Copolymer-Derived Nanospheres. Biomacromol. 2005, 6, 2726-2731.
10. Deming, T. J. Methodologies for Preparation of Synthetic Block Copolypeptides: Materials with Future Promise in Drug Delivery. Adv. Drug Deliv. Rev., 2002, 54, 1145-1155.
11. Lacerda, L.; Bianco, A.; Prato, M.; Kostarelos, K. Carbon Nanotubes as Nanomedicines: From Toxicology to Pharmacology. Adv. Drug Deliv. Rev., 2006, 58, 1460-1470.
12. Langer, R. New Methods of Drug Delivery. Science, 1990, 249, 1527-1533.
13. Peer, D.; Karp, J. M.; Hong, S.; Farokhzad, O. C.; Margalit, R.; Langer, R. Nanocarriers as an Emerging Platform for Cancer Therapy. Nat. Nanotechnol., 2007, 2, 751-760.
14. Kam N. W. S.; O'Connell, M.; Wisdom, J. A.; Dai, H. Carbon Nanotubes as Multifunctional Biological Transporters and Near-Infrared Agents for Selective Cancer Cell Destruction. Proc. Nat. Acad. Sci.-USA 2005, 102, 11600-11605.
15. Liu, Z.; Robinson, J. T.; Sun, X.; Dai, H. PEGylated Nanographene Oxide for Delivery of Water-Insoluble Cancer Drugs. J. Am. Chem. Soc. 2008, 130, 10876-10877.
16. Gruen, D. M. Nanocrystalline Diamond Films. Annu. Rev. Mater. Sci., 1999, 29, 211-259.
17. Petrov, I. L.; Shenderova, I. L. Ultra Nanocrystalline Diamond: Synthesis, Properties and Applications; Shenderova, O. A.; Gruen, D. M., Eds.; William Andrew Publishing New York, 2006; pp 529-550.
18. Yeap, W. S.; Tan, Y. Y.; Loh, K. P. Using Detonation Nanodiamond for the Specific Capture of Glycoproteins. Anal. Chem., 2008, 80, 4659-4665.
19. Kruger, A. Hard and Soft: Biofunctionalized Diamond. Angew. Chem. Int. Ed., 2006, 45, 6426-6427.
20. Huang, L.-C. L.; Chang, H.-C. Adsorption and Immobilization of Cytochrome c on Nanodiamonds. Langmuir, 2004, 20, 5879-5884.
21. Ushizawa, K.; Sato, Y.; Mitsumori, T.; Machinami, T.; Ueda, T.; Ando, T. Covalent Immobilization of DNA on Diamond and its Verification by Diffuse Reflectance Infrared Spectroscopy. Chem. Phys. Lett., 2002, 351, 105-108.
22. Krüger, A.; Kataoka, F.; Ozawa, M.; Fujino, T.; Suzuki, Y.; Aleksenskii, A. E.; Vul, A. Y.; Ōsawa, E. Unusually Tight Aggregation in Detonation Nanodiamond: Identification and Disintegration. Carbon, 2005, 43, 1722-1730.
23. Bondar, V. S.; Puzyr, A. P. Nanodiamonds for Biological Investigations. Phys. Solid State, 2004, 46, 716-719.
24. Ozawa, M.; Inaguma, M.; Takahashi, M.; Kataoka, F.; Krüger, A.; Ōsawa, E. Preparation and Behavior of Brownish, Clear Nanodiamond Colloids. Adv. Mater., 2007, 19, 1201-1206.
25. Kossovsky, N.; Gelman, A.; Hnatyszyn, H. J.; Rajguru, S.; Garrell, R. L.; Torbati, S.; Freitas, S. S. F.; Chow, G.-M. Surface-Modified Diamond Nanoparticles as Antigen Delivery Vehicles. Bioconjugate. Chem., 1995, 6, 507-511.
26. Huang, H.; Pierstorff, E.; Ōsawa, E.; Ho, D. Active Nanodiamond Hydrogels for Chemotherapeutic Delivery. Nano Lett., 2007, 7, 3305-3314.
27. Huang, H.; Pierstorff, E.; Ōsawa, E.; Ho, D. Protein-Mediated Assembly of Nanodiamond Hydrogels into a Biocompatible and Biofunctional Multilayer Nanofilm. ACS Nano, 2008, 2, 203-212.
28. Lam, R.; Chen, M.; Pierstorff, E.; Huang, H.; Osawa, E.; Ho, D. Nanodiamond-Embedded Microfilm Devices for Localized Chemotherapeutic Elution. ACS Nano 2008, 2, 2095-2102.
29. Huang, H.; Chen, M.; Bruno, P.; Lam, R.; Robinson, E.; Gruen, D.; Ho, D. Ultrananocrystalline Diamond Thin Films Functionalized with Therapeutically Active Collagen Networks. J. Phys. Chem. B 2009, 113, 2966-2971.
30. Dolmatov, V. Y. Detonation Synthesis Ultradispersed Diamonds: Properties and Applications. Russ. Chem. Rev., 2001, 70, 607-626.
31. Schrand, A. M.; Huang, H.; Carlson, C.; Schlager, J. J.; Ōsawa, E.; Hussain, S. M.; Dai, L. Are Diamond Nanoparticles Cytotoxic? J. Phys. Chem. B, 2007, 111, 2-7.
32. Liu, K.-K.; Cheng, C.-L.; Chang, C.-C.; Chao, J.-I. Biocompatible and Detectable Carboxylated Nanodiamond on Human Cell. Nanotechnology, 2007, 18, 325102.
33. Yu, S.-J.; Kang, M.-W.; Chang, H.-C.; Chen, K.-M.; Yu, Y.-C. Bright Fluorescent Nanodiamonds: No Photobleaching and Low Cytotoxicity. J. Am. Chem. Soc., 2005, 127, 17604-17605.
34. Härtl, A.; Schmich, E.; Gamido, J. A.; Hernando, J.; Catharino, S. C. R.; Walter, S.; Feulner, P.; Kromka, A.; Steinmüller, D.; Stutzmann, M. Protein-Modified Nanocrystalline Diamond Thin Films for Biosensor Applications. Nat. Mater., 2004, 3, 736-742.
35. Yang, W.; Auciello, O.; Butler, J. E.; Cai, W.; Carlisle, J. A.; Gerbi, J. E.; Gruen, D. M.; Knickerbocker, T.; Lasseter, T. L.; Russell, Jr., et al. DNA-Modified Nanocrystalline Diamond Thin-Films as Stable, Biologically Active Substrates. Nat. Mater., 2002, 1, 253-257.
36. Neugart, F.; Zappe, A.; Jelezko, F.; Tietz, C.; Boudou, J. P.; Krueger, A.; Wrachtrup, J. Dynamics of Diamond Nanoparticles in Solution and Cells. Nano Lett. 2007, 7, 3588-3591.
37. Fisher, B.; Costantino, J. P.; Wickerham, D. L.; Redmond, C. K.; Kavanah, M.; Cronin, W. M,; et al. Tamoxifen for Prevention of Breast Cancer: Report of the National Surgical Adjuvant Breast and Bowel Project P-1 Study. J. Natl. Cancer Inst. 1998, 90, 1371-1388.
38. Fisher, B.; Dignam, J.; Wolmark, N.; Wickerham, D. L.; Fisher, E. R.; Mamounas, E. et al. Tamoxifen in Treatment of Intraductal Breast Cancer: National Surgical Adjuvant Breast and Bowel Project B-24 Randomised Controlled Trial. Lancet 1999, 353,1993-2000.
39. Taylor, C. M.; Blanchard, B.; Zava, D. T. Estrogen Receptor-mediated and Cytotoxic Effects of the Antiestrogens Tamoxifen and 4-Hydroxytamoxifen. Cancer Res. 1984, 44, 1409-1414.
40. Gauduchon, J.; Gouilleux, F.; Maillard, S.; Marsaud, V.; Renoir, J.-M.; Sola, B.-Hydroxytamoxifen Inhibits Proliferation of Multiple Myeloma Cells In vitro through Down-Regulation of c-Myc, Up-Regulation of p27^{Kip1}, and Modulation of Bc1-2 Family Members. Clin. Canc. Res. 2005, 11, 2345-2354.
41. Kircheis, R.; Wightman, L.; Wagner, E. Design and Gene Delivery Activity of Modified Polyethylenimines. Advanced Drug Delivery Reviews 2001, 53, 341-358.
42. Bettinger, T.; Remy, J-S.; Erbacher, P. Size Reduction of Galactosylated PEI/DNA Complexes Improves Lectin-Mediated Gene Transfer into Hepatocytes. Bioconjugate Chem. 1999, 10, 558-561.

### References for Part III

### (Alkaline-Sensitive Nanodiamond-Protein Complexes) and Example 2

1. Mochalin V N, Gogotsi Y. Wet Chemistry Route to Hydrophobic Blue Fluorescent Nanodiamond. J Am Chem Soc 2009; 131(13):4594-5.
2. Chang Y-R, Lee H-Y, Chen K, Chang C-C, Tsai D-S, Fu C-C, Lim T-S, Tzeng Y-K, Fang C-Y, Han C-C, Chang H-C, Fann W. Mass production and dynamic imaging of fluorescent nanodiamonds. Nat Nanotechnol 2008; 3(5):284-8.
3. Fu C-C, Lee H-Y, Chen K, Lim T-S, Wu H-Y, Lin P-K, Wei P-K, Tsao P-H, Chang H-C, Fann W. Characterization and application of single fluorescent nanodiamonds as cellular biomarkers. Proc Nat Acad Sci USA 2007; 104(3):727-32.
4. Deming T J. Methodologies for preparation of synthetic block copolypeptides: materials with future promise in drug delivery. Adv Drug Deliv Rev 2002; 54(8):1145-55.
5. Zhang L, Chan J M, Gu F X, Rhee J-W, Wang A Z, Radovic-Moreno A F, Alexis F, Langer R, Farokhzad O C. Self-assembled lipid-polymer hybrid nanoparticles: a robust drug delivery platform. ACS Nano 2008; 2(8):1696-702.
6. Farokhzad O C, Langer R. Impact of Nanotechnology on Drug Delivery. ACS Nano 2009; 3(1):16-20.
7. Xu P, Gullotti E, Tong L, Highley C B, Errabelli D R, Hasan T, Cheng J-X, Kohane D S, Yeo Y. Intracellular drug delivery by poly(lactic-co-glycolic acid) nanoparticles, revisited. Mol Pharm 2009; 6(1):190-201.
8. Huang H, Pierstorff E, Osawa E, Ho D. Active Nanodiamond Hydrogels for Chemotherapeutic Delivery. Nano Lett 2007; 7(11):3305-14.
9. Lam R, Chen M, Pierstorff E, Huang H, Osawa E, Ho D. Nanodiamond-Embedded Microfilm Devices for Localized Chemotherapeutic Elution. ACS Nano 2008; 2(10):2095-102.
10. Pathak P, Meziani M J, Desai T, Foster C, Diaz J A, Sun Y P. Supercritical fluid processing of drug nanoparticles in stable suspension. J Nanosci Nanotechnol 2007; 7(7):2542-5.
11. Cheong S-J, Lee C-M, Kim S-L, Jeong H-J, Kim E-M, Park E-H, Kim D W, Lim S T, Sohn M-H. Superparamagnetic iron oxide nanoparticles-loaded chitosan-linoleic acid nanoparticles as an effective hepatocyte-targeted gene delivery system. Int J Pharm 2009; 372(1-2):169-76.
12. Dobson J. Gene therapy progress and prospects: magnetic nanoparticle-based gene delivery. Gene Ther 2006; 13(4):283-7.
13. Panyam J, Labhasetwar V. Biodegradable nanoparticles for drug and gene delivery to cells and tissue. Adv Drug Deliv Rev 2003; 55(3):329-47.
14. Allen T M, Cullis P R. Drug Delivery Systems: Entering the Mainstream. Science 2004; 303(5665):1818-22.
15. Gwinn M R, Vallyathan V. Nanoparticles: Health Effects—Pros and Cons. Environ Health Perspect 2006; 114(12):1818-25.
16. Niemeyer C M. Nanoparticles, proteins, and nucleic acids: biotechnology meets material science. Angew Chem Int Ed 2001; 40(22):4128-58.
17. Gelperina S, Kisich K, Iseman M D, Heifets L. The Potential Advantages of Nanoparticle Drug Delivery Systems in Chemotherapy of Tuberculosis. Am J Resp Crit Care 2005; 172(12):1487-90.
18. Hilder T A, Hill J M. Carbon nanotubes as drug delivery nanocapsules. Curr Appl Phys 2007; 8(3-4):258-61.
19. Ajima K, Yudasaka M, Murakami T, Maigne A, Shiba K, Iijima S. Carbon Nanohorns as Anticancer Drug Carriers. Mol Pharm 2005; 2(6):475-80.
20. Cevc G, Richardsen H. Lipid vesicles and membrane fusion. Adv Drug Deliv Rev 1999; 38(3):207-32.
21. Mirsa RDK. Magnetic nanoparticle carrier for targeted drug delivery: perspective, outlook and design. J Mater Sci Technol 2008; 24(9):1011-9.
22. Huang H, Chen M, Bruno P, Lam R, Robinson E, Gruen D, Ho D. Ultrananocrystalline Diamond Thin Films Functionalized with Therapeutically Active Collagen Networks. J Phys Chem B 2009; 113(10):2966-71.
23. Behler K D, Stravato A, Mochalin V, Korneva G, Yushin G, Gogotsi Y. Nanodiamond-Polymer Composite Fibers and Coatings. ACS Nano 2009; 3(2):363-9.
24. Hanson J A, Chang C B, Graves S M, Li Z, Mason T G, Deming T J. Nanoscale double emulsions by single-component block copolypeptides. Nature 2008; 455(7209):85-8.
25. Auguste D T, Furman K, Wong A, Fuller J, Armes S P, Deming T J, Langer R. Triggered release of siRNA from poly(ethylene glycol)-protected, pH-dependent liposomes. J Control Release 2008; 130(3):266-74.
26. Yeo Y, Ito T, Bellas E, Highley C B, Marini R, Kohane D S. In Situ Cross-linkable Hyaluronan Hydrogels Containing Polymeric Nanoparticles for Preventing Postsurgical Adhesions. Ann Surg 2007; 245(5):819-24.
27. Pathak P, Meziani M J, Desai T, Sun Y P. Nanosizing drug particles in supercritical fluid processing. J Am Chem Soc 2004; 126(35):10842-3.
28. Huang H, Pierstorff E, Osawa E, Ho D. Protein-Mediated Assembly of Nanodiamond Hydrogels into a Biocompatible and Biofunctional Multilayer Nanofilm. ACS Nano 2008; 2(2):203-12.
29. Yeap W S, Chen S, Loh K P. Detonation Nanodiamond: An Organic Platform for the Suzuki Coupling of Organic Molecules. Langmuir 2009; 25(1):185-91.
30. Panessa-Warren B J, Warren J B, Wong S S, Misewich J A. Biological cellular response to carbon nanoparticle toxicity. J Phys: Condens Matter 2006; 18(33):52185-5201.
31. Puzyr A P, Baron A V, Purtov K V, Bortnikov E V, Skobelev N N, Mogilnaya O A, Bondar V S. Nanodiamonds with novel properties: A biological study. Diamond Relat Mater 2007; 16(12):2124-8.
32. Schrand A M, Huang H, Carlson C, Schlager J J, Osawa E, Hussain S M, Dai L. Are Diamond Nanoparticles Cytotoxic? J Phys Chem B 2007; 111(1):2-7.
33. Zhao W, Xu J-J, Qiu Q-Q, Chen H-Y. Nanocrystalline diamond modified gold electrode for glucose biosensing. Biosens Bioelectron 2006; 22(5):649-55.
34. Huang LCL, Chang H-C. Adsorption and Immobilization of Cytochrome c on Nanodiamonds. Langmuir 2004; 20(14):5879-84.
35. Cao H, Urban J F, Anderson R A. Insulin Increases Tristetraprolin and Decreases VEGF Gene Expression in Mouse 3T3-L1 Adipocytes. Obesity 2008; 16(6):1208-18.
36. Adochio R, Leitner J W, Hedlund R, Draznin B. Rescuing 3T3-L1 Adipocytes from Insulin Resistance Induced by Stimulation of Akt-Mammalian Target of Rapamycin/p70 S6 Kinase (S6K1) Pathway and Serine Phosphorylation of Insulin Receptor Substrate-1: Effect of Reduced Expression of p85α Subunit of Phosphatidylinositol 3-Kinase and S6K1 Kinase. Endocrinology 2009; 150(3):1165-73.
37. Speller C V, Meot-Ner M. The ionic hydrogen bond and ion solvation. 3. Bonds involving cyanides. Correlations with proton affinities. J Phys Chem 1985; 89(24):5217-22.
38. Farías R N, Viñals AEL, Posse E, Morero R D. Relationship between isoelectric point of native and chemically modified insulin and liposomal fusion. Biochem J 1989; 264(1):285-7.
39. Feng L, Andrade J D. Protein adsorption of low-temperature isotropic carbon: I. Protein conformational change probed by differential scanning calorimetry. J Biomed Mater Res 1994; 28(6):735-43.
40. Lida K T, Suzuki H, Sone H, Shimano H, Toyoshima H, Yatoh S, Asano T, Okuda Y, Yamada N. Insulin Inhibits Apoptosis of Macrophage Cell Like, THP-1 Cells, via Phosphatidylinositol-3-Kinas-Dependent Pathway. Arterioscler Thromb Vasc Biol 2002; 22(3):380-6.
41. Greenway S E, Filler L E, Greenway F L. Topical insulin in wound healing: a randomised, double-blind, placebo-controlled trial. J Wound Care 1999; 8(10):526-8.
42. Liu Y, Petreaca M, Yao M, Martins-Green M. Cell and molecular mechanisms of keratinocyte function stimulated by insulin during wound healing. BMC Cell Biol 2009; 10(1).
43. Pierre E J, Barrow R E, Hawkins H K, Nguyen T T, Sakurai Y, Desai M, Wolfe R R, Herndon DN. Effects of insulin on wound healing. J Trauma 1998; 44(2):342-5.
44. Liu Y, Zhang X, Zhang Z, Fang P Y, Xu W S. Effects of topical application of insulin on the wound healing in scalded rats. Chinese J Burns 2004; 20(2):98-101.
45. Zhang X J, Wu X, Wolf S E, Hawkins H K, Chinkes D L, Wolfe R R. Local insulin-zinc injection accelerates skin donor site wound healing. J Surg Res 2007; 142(1):90-6.
46. Schneider L A, Korber A, Grabbe S, Dissemond J. Influence of pH on wound-healing: a new perspective for wound-therapy? Arch Dermatol Res 2007; 298(9):413-20.
47. Osti E. Skin pH variations from the acute phase to re-epithelialization in burn patients treated with new materials (Burnshield, semipermeable adhesive film, Dermasilk, and Hyalomatrix). Non-invasive preliminary experimental clinical trial. Ann Burns and Fire Disasters 2007; 21(2):73-7.

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A composition comprising a soluble complex, wherein said soluble complex comprises:
a) a nanodiamond particle comprising one or more surface carboxyl groups; and
b) a therapeutic agent, wherein said therapeutic agent is inherently water-insoluble or poorly water soluble, wherein said therapeutic agent is adsorbed to said nanodiamond particle to form said soluble complex, wherein said soluble complex is soluble in water.

2. A method of making a soluble complex comprising: mixing a nanodiamond particle with a therapeutic agent in the presence of an acid solution such that said therapeutic agent adsorbs to said nanodiamond particle thereby forming a soluble complex, wherein said therapeutic agent is inherently water-insoluble or poorly water soluble.

3. The method of claim 2, wherein said acid solution comprises acetic acid.

4. A composition comprising a nanodiamond-nucleic acid complex, wherein said complex comprises:
a) nanodiamond particles comprising one or more surface polyethyleneimine molecules; and
b) nucleic acid molecules, wherein said nucleic acid molecules and said nanodiamond particles form a nanodiamond-nucleic acid complex.

5. The composition of claim 4, wherein said nanodiamond particles and said nucleic acid molecules form said nanodaimond-nucleic acid complex via attraction of positive charges on said nanodaimond particles and negative charges on said nucleic acid molecules.

6. The composition of claim 4, wherein said nucleic acid molecules in said nanodiamond-nucleic acid complex are attached to said nanodiamond particles such that they are released upon cellular introduction.

7. The composition of claim 4, wherein said polyethyleneimine molecules are low molecular weight polyethyleneimine molecules.

8. A composition comprising an alkaline-sensitive nanodiamond-protein complex, wherein said alkaline-sensitive nanodiamond complex comprises:
a) a nanodiamond particle comprising one or more surface carboxyl or hydroxyl groups; and
b) a protein, wherein said protein is adsorbed to said nanodiamond particle to form said alkaline-sensitive nanodiamond-protein complex, wherein said protein is configured to desorb from said nanodiamond particle only under sufficiently alkaline conditions.

9. The composition of claim 8, wherein said alkaline conditions are a pH of at least 9.0.

10. The composition of claim 8, wherein said alkaline conditions are a pH of at least 10.0.

11. A composition comprising a soluble complex, wherein said soluble complex comprises:
a) a nanodiamond particle; and
b) a therapeutic agent, wherein said therapeutic agent comprises an anthracycline-class compound or tetracycline-class compound.

12. The composition of claim 11, wherein said anthracycline-class compound or tetracycline-class compound is selected from: daunorubicin, doxorubicin, epirubicin, idarubicin, valrubicin, mitoxantrone, tetracycline, chlortetracycline, oxytetracycline, demeclocycline, doxycycline, lymecycline, meclocycline, methacycline, minocycline, and rolitetracycline.

13. The composition of claim 11, wherein said anthracycline-class compound or tetracycline-class compound in said soluble complex is released upon cellular introduction.
